(19) United States
(12) Patent Application Publication
(10) Pub. No.: US 2026/0124253 A1
(43) Pub. Date: May 7, 2026

(54) METHOD FOR ENHANCING TRANSFERRIN RECEPTOR-BASED TRANSCYTOSIS ACROSS THE BLOOD BRAIN BARRIER

(71) Applicants: Nankai University, Nankai District (CN); MedBio Reference Laboratories, Inc., Wheaton, MD (US)

(72) Inventors: Lei Wang, Tianjin, P.R. (CN); Huamin Henry Li, Ellicott City, MD (US); Bin Liu, Tianjin, P.R (CN); Lili Wan, Clarksville, MD (US); Lu Feng, Tianjin, P.R. (CN); Shaobin Hou, Honolulu, HI (US); J. Joanna Yu, Ellicott City, MD (US); Joseph Chiao, Allentown, NJ (US)

(73) Assignees: Nankai University, Nankai District (CN); MedBio Reference Laboratories, Inc., Wheaton, MD (US)

(21) Appl. No.: 18/940,574

(22) Filed: Nov. 7, 2024

(51) Int. Cl. A61K 35/74; A61K 38/17

(52) U.S. Cl. CPC A61K 35/74; A61K 38/1709

(57) ABSTRACT

Methods for enhancing transcytosis comprising modulating expression of Vesicle-associated membrane protein 3 (VAMP3) and syntaxin 4 (STX-4). Composition for increasing expression of VAMP3 and/or STX-4 in HBMECs and compositions comprising target molecules in a form suitable for transcytosis across the blood brain barrier.

### REFERENCE TO A SEQUENCE LISTING

In accordance with 37 CFR § 1.831-1.835 and 37 CFR § 1.77(b)(5), this specification includes a Sequence Listing as part of the application. The Sequence Listing is provided as an XML file named “549922US_110724_ST26.xml” in compliance with WIPO Standard ST.26. This file is 12,754 bytes in size and was generated on Nov. 7, 2024. The information recorded in the Sequence Listing XML file is identical to the sequence information described in the application as filed. The entire contents of the Sequence Listing are hereby incorporated by reference.

### BACKGROUND OF THE INVENTION

Field of the Invention. The invention pertains broadly to the field of medicine and especially to the fields of drug or biologic delivery, cell biology, neuroscience, and pharmacology.

Description of Related Art. The blood-brain barrier (BBB) is a highly selective semipermeable border that separates the circulating blood from the brain and extracellular fluid in the central nervous system. Bacterial meningitis occurs when pathogenic bacteria penetrate this barrier and invade the brain, leading to inflammation of the meninges and potentially severe neurological complications.

The BBB is composed of human brain microvascular endothelial cells (HBMECs) and is an essential gatekeeper for the central nervous system (CNS). It uniquely separates brain's internal milieu from the circulating blood. Kim K S (2008) Mechanisms of microbial traversal of the blood-brain barrier. Nat Rev Microbiol 6(8):625-634.

With the features of numerous intercellular tight-junctions and low rates of transcytosis, the BBB is characterized by a very low permeability for biomolecules, microorganisms, and toxins in order to protect and regulate the metabolism of the brain and maintain the neural microenvironment. Sweeney M D, Zhao Z, Montagne A, Nelson A R, & Zlokovic B V (2019) Blood-Brain Barrier: From Physiology to Disease and Back. Physiol Rev 99(1):21-78; Langen U H, Ayloo S, & Gu C (2019) Development and Cell Biology of the Blood-Brain Barrier. Annu Rev Cell Dev Biol 35:591-613.

To guarantee the proper functioning of the brain, several low-rate transcytosis pathways across the BBB are employed under tight regulation to ensure an adequate supply of ions, nutrients, and essential signaling molecules required by nervous tissue. Zhao Z & Zlokovic B V (2020) Therapeutic TVs for Crossing Barriers in the Brain. Cell 182(2):267-269.

Among these pathways, transcytosis that is mediated by the transferrin receptor (TfR) provides an important route for delivering iron to the brain, which is essential for multiple neurological functions. Zuchero Y J, et al. (2016) Discovery of Novel Blood-Brain Barrier Targets to Enhance Brain Uptake of Therapeutic Antibodies. Neuron 89(1):70-82; Preston J E, Joan Abbott N, & Begley D J (2014) Transcytosis of macromolecules at the blood-brain barrier. Adv Pharmacol 71:147-163.

The impermeable nature of the BBB poses a significant challenge to the uptake of therapeutic agents into the brain. Andreone B J, et al. (2017) Blood-Brain Barrier Permeability Is Regulated by Lipid Transport-Dependent Suppression of Caveolae-Mediated Transcytosis. Neuron 94(3):581-594 e585.

The TfR transcytosis-mediated delivery system has been extensively utilized for the transport of drugs across the BBB and is considered one of the most promising brain delivery approaches (Zhao, 2020 #4969). Terstappen G C, Meyer A H, Bell R D, & Zhang W (2021) Strategies for delivering therapeutics across the blood-brain barrier. Nat Rev Drug Discov 20(5):362-383; Fishman J B, Rubin J B, Handrahan J V, Connor J R, & Fine R E (1987) Receptor-mediated transcytosis of transferrin across the blood-brain barrier. J Neurosci Res 18(2):299-304.

Several TfR-targeting antibodies and antibody-drug conjugates have demonstrated encouraging outcomes for brain delivery in clinical trials. Terstappen G C, Meyer A H, Bell R D, & Zhang W (2021) Strategies for delivering therapeutics across the blood-brain barrier. Nat Rev Drug Discov 20(5):362-383; Yu Y J, et al. (2014) Therapeutic bispecific antibodies cross the blood-brain barrier in nonhuman primates. Sci Transl Med. 2014 Nov. 5; 6(261):261ra154; Rawal S U, Patel B M, & Patel M M (2022) New Drug Delivery Systems Developed for Brain Targeting. Drugs 82(7):749-792.

However, efficiency remains low despite significant efforts that have been made to improve the TfR transcytosis-mediated delivery system through methods such as antibody engineering. Zhou Q H, et al. (2011), Receptor-mediated Abeta amyloid antibody targeting to Alzheimer's disease mouse brain. Mol Pharm 8(1):280-285; Yu Y J, et al. (2014); Therapeutic bispecific antibodies cross the blood-brain barrier in nonhuman primates; Sci Transl Med. 2014 Nov. 5; 6(261):261ra154; Ullman J A-O, et al. (2020). Brain delivery and activity of a lysosomal enzyme using a blood-brain barrier transport vehicle in mice Sci Transl Med. 2020 May 27; 12(545):eaay1163. There remains a significant need for a more efficient method of TfR transcytosis across the BBB. Advantages of improving the efficiency of TfR transcytosis include but are not limited to enhanced and less invasive delivery of drugs and other therapeutics to the brain, improved pharmacokinetics, more targeted treatment of neurodegenerative diseases, and enhanced or more specific imaging of the brain.

### BRIEF SUMMARY OF THE INVENTION

The inventors have found that three major meningitis-causing bacterial pathogens with different evolutionary histories have employed the same or a similar mechanism to cross the BBB. These pathogens include neonatal meningitis Escherichia coli (NMEC), Streptococcus pneumoniae and group B Streptococcus, which infect and invade the brain from the blood stream. They all activate the same fusion process of bacteria-containing vesicles (BCVs) with TfR vesicles within HBMECs, thereby hijacking TfR transcytosis to penetrate the BBB. Cheng Z, et al. (2023) Pathogenic bacteria exploit transferrin receptor transcytosis to penetrate the blood-brain barrier. Proc Natl Acad Sci USA 120(39):e2307899120. This finding further demonstrates that utilizing TfR transcytosis for the transport of drugs across the BBB is an exciting and potentially viable option.

In work disclosed herein, the inventors firstly revealed that VAMP3 present on TfR vesicles and TfR-NMEC vesicles interacts with syntaxin 4 at the basolateral membrane of HBMECs, mediating the transcytosis of Tf and NMEC.

Silence or mutation of VAMP3 and syntaxin 4 reduced the transcytosis of Tf and NMEC across the BBB in vitro and in mice.

It was discovered that NMEC infection induces VAMP3 and syntaxin 4 expression via the activation of TLR4-TRAM-TRIF-TRAF3-IKK-IRF3 regulatory pathway by LPS. Furthermore, the inventors showed that high overexpression of VAMP3 or syntaxin 4 promoted the fusion of TfR vesicles and TfR-NMEC vesicles with the basolateral membrane of HBMECs, and thus increased the efficiency of TfR mediated transcytosis.

This work provides essential insights for developing strategies for the treatment of meningitis caused by NMEC and improved drug deliver into the brain.

One aspect of the invention is a method for enhancing TfR transcytosis through the BBB.

Another aspect of the invention is a method for modulating transcytosis through the BBB by increasing or decreasing the expression of VAMP3 or STX-4 proteins or by increasing or decreasing levels of VAMP3 or STX-4 in HBMECs, their vesicles, or their other components.

Embodiments include but are not limited to the following.
1. A method for modulating transcytosis of a target molecule in a cell comprising modulating expression of, or the amount of Vesicle-associated membrane protein 3 (VAMP3) and/or expression of, or amount of, Syntaxin 4 (STX-4). In one preferred embodiments, the transcytosis is medicated by binding of a target molecule or target molecule-conjugate to a transferrin receptor on the lumina side of a Human Brain Microvascular Endothelial Cell (HBMEC).
2. The method of embodiment 1, wherein the cell is a Human Brain Microvascular Endothelial Cell (HBMEC).
3. The method of embodiment 1 or 2, wherein VAMP3 expression or cellular levels of VAMP3 are increased in a HBMEC compared to otherwise similar HBMCs not treated to increase VAMP3 expression or VAMP3 level.
4. The method of any one of embodiments 1-3, wherein VAMP3 expression or VAMP3 level is increased by administering a drug, peptide, polypeptide or polynucleotide that increases VAMP3 expression.
5. The method of any one of embodiments 1-4, wherein VAMP3 expression is increased by administering live, attenuated, or dead neonatal meningitis Escherichia coli (NMEC), Streptococcus pneumoniae, or group B Streptococcus, LPS, or components thereof which increase expression of VAMP3.
6. The method of any one of embodiments 1-5, wherein level of VAMP 3 in HBMECs is increased by transiently or permanently transforming the HBMECs with a nucleic acid encoding VAMP3.
7. The method of any one of embodiments 1-6, wherein VAMP3 level in HBMECs is increased by loading the HBMECs with exogenous VAMP3.
8. The method of any one of embodiments 1-7, wherein VAMP3 level in HBMECs is increased by loading vesicles that fuse with the HBMECs with exogenous VAMP3.
9. The method of any one of embodiments 1-8, wherein said transcytosis comprises movement of the target molecule that is an antibody or other protein molecule, or a conjugate thereof, from the blood compartment to a brain compartment.
10. The method of any one of embodiments 1-9, wherein said transcytosis comprises movement of the target molecule that is a drug or pharmaceutical, or a conjugate thereof, from the blood compartment to a brain compartment.
11. The method of any one of embodiments 1-10, wherein STX-4 expression or cellular level of STX-4 are increased in a HBMEC compared to otherwise similar HBMCs not treated to increase STX-4 expression or STX-4 level.
12. The method of any one of embodiments 1-11, wherein STX-4 expression or STX-4 level is increased by administering a drug, peptide or polynucleotide that increases STX-4 expression.
13. The method of any one of embodiments 1-12, wherein STX-4 expression is increased by administering live, attenuated, or dead neonatal meningitis Escherichia coli (NMEC), Streptococcus pneumoniae, or group B Streptococcus, LPS, or components thereof which increases expression of STX-4.
14. The method of any one of embodiments 1-13, wherein level of STX-4 in HBMECs is increased by transiently or permanently transforming the HBMECs with a nucleic acid encoding STX-4.
15. The method of any one of embodiments 1-14, wherein STX-4 level in HBMECs is increased by loading the HBMECs with exogenous STX-4.
16. The method of any one of embodiments 1-15, wherein STX-4 level in HBMECs is increased by loading vesicles that fuse with the HBMECs with exogenous STX-4.
17. The method of any one of embodiments 1-16, wherein said transcytosis comprises movement of the target molecule that is an antibody or other protein molecule, or a conjugate thereof, from the blood compartment to a brain compartment.
18. The method of any one of embodiments 1-17, wherein said transcytosis comprises movement of the target molecule that is a drug or pharmaceutical, or a conjugate thereof, from the blood compartment to a brain compartment.
19. The method of any one of embodiments 1-18, wherein both VAMP3 and STX-4 expression or levels are increased compared to an otherwise similar HBMCs not treated to increase VAMP3 expression or VAMP3 level and STX-4 expression or STX-4 level.
20. The method of embodiment 1 or 2, wherein the level of, or expression of, VAMP3 is decreased.
21. The method of embodiment 20, wherein VAMP3 expression is decreased by administering siRNA that targets mRNA encoding VAMP3.
22. The method of embodiment 20 or 21 wherein VAMP3 expression is decreased by administering an antibody or other agent that binds to VAMP3.
23. The method of embodiment 1 or 2, wherein the level of or expression of STX-4 is decreased.
24. The method of embodiment 23, wherein STX-4 expression is decreased by administering siRNA that targets mRNA encoding STX-4.
25. The method of embodiment 23 or 24, wherein STX-4 expression is decreased by administering an antibody or other agent that binds to STX-4.
26. The method of embodiment 1 or 2, wherein the levels of both VAMP3 and STX-4 are decreased.
27. A method for treating a subject for a brain disease or disorder in need of transcytosis of a therapeutic agent into the brain, comprising increasing a level of VAMP3 and/or increasing a level of STX-4 in HBMECs or in vesicles that can fuse with HBMECs.
28. The method of claim 27, wherein the subject has a brain disease or disorder in need of transcytosis of a drug or biologic into the brain.
29. A method for treating a subject for a brain disease or disorder in need of a reduction of transcytosis into the brain comprising decreasing a level of VAMP3 and/or a level of STX-4 in HBMECs or in vesicles that can fuse with HBMECs.
30. The method of claim 29, wherein the subject has a brain disorder or disease in need of a reduction of transcytosis of a virus, bacterium, fungus or other infectious agent into the brain.
31. The method of embodiment 29, wherein the subject has a brain disorder or disease in need of a reduction of transcytosis of a drug, metal, antibody or other biologic or other chemical agent into the brain.
32. The method of embodiment 29, wherein the subject has viral or bacterial meningitis.
33. The method of embodiment 29, wherein the subject is at risk of or has neonatal meningitis caused by, or associated with, infection or exposure to Escherichia coli (NMEC), Streptococcus pneumoniae or group B Streptococcus.
34. A composition comprising
an agent that increases the expression of VAMP3 and/or STX4-when administered to HBMEC cells in combination with a target molecule or target molecule conjugate or complex that binds to TfR and initiates transcytosis through a blood brain barrier comprising said HBMECs.
35. A composition comprising:
VAMP3 and/or STX-4 in combination with a target molecule or target molecule conjugate or complex, such as a pharmaceutical, drug, peptide, polypeptide, antibody, or conjugates or complexes thereof, in a form suitable for uptake or incorporation HBMECs or incorporation into vesicles comprising TfR.

A more complete appreciation of the disclosure and many of the attendant advantages thereof will be readily obtained as the same becomes better understood by reference to the following detailed description when considered in connection with the accompanying drawings below. The figures described below show the effects of overexpressing or silencing VAMP3 or STX-4 on Tf (transferrin) or on NMEC transcytosis across HBMECs. For instance, as shown in FIGS. 3C and 5I and their brief descriptions the overexpression of VAMP3 increased transcytosis efficiency by 2.46 to 3.79 fold in in vitro transwell assay; and as shown in FIGS. 3F and 5J and their brief descriptions their overexpression of STX-4 increased transcytosis efficiency by 2.02 to 3.23 fold in in vitro transwell assay.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1C. VAMP3 is involved in the transcytosis of Tf through HBMECs.

FIG. 1A. Spectrophotometric fluorescence intensity measurement of FITC-Tf transcytosis from the apical side to the basolateral side of HBMECs transfected with control siRNA or siRNA targeting VAMP1, VAMP2, VAMP3, VAMP4, VAMP7 and VAMP8. The excitation is 495 nm and emission is 518 nm. (n=3 independent experiments). This figure shows that silencing VAMP3 by siRNA significantly decreased FITC-Tf transcytosis by 3.16-fold (31.7% vs 100%) in in vitro Transwell assay (Spectrophotometric fluorescence intensity data 1127 vs 3561).

FIG. 1B. Colocalization of VAMP3 (red) with TfR (green) in HBMECs. Cell nuclei were stained with DAPI (blue). Quantification of colocalization was shown by calculating the Pearson correlation coefficients (PC), Mander's coefficient M1 (fraction of VAMP3 colocalized with TfR) and M2 (fraction of TfR colocalized with VAMP3). Colocalization of VAMP3 with TfR indicates localization of VAMP3 to TfR vesicles. There were n=10 random areas per group from three independent experiments. Scale bar, 2 m (lower right).

FIG. 1C. Effect of VAMP3 deficiency on transcytosis of Tf-biotin across the BBB in vivo. WT (wild type) mice and gene-deficient VAMP3^{-/-} (VAMP3 KO) mice (n=6) received Tf-biotin via the tail vein. 1 μL cerebrospinal fluid from each mouse was collected after 4 hours for kit detection. VAMP3 knock-out mice did not transport Tf-biotin across the BBB. This figure shows that FITC-Tf transcytosis across the BBB was undetected in VAMP3^{-/-} mice in vivo. Data are presented as the means±SD, *P<0.05; **P<0.01; ***P<0.001, ns, not significant. One-way ANOVA (1A), Mann-Whitney U test (1C).

FIGS. 2A-2G. VAMP3 interacts with syntaxin 4 (STX-4) at the basolateral membrane of HBMECs.

FIGS. 2A and 2B. Co-immunoprecipitation assays of VAMP3 and STX-4 in HBMECs. These results show that VAMP3 was efficiently coimmunoprecipitated with syntaxin 4. Hsp60 and Hsp70, loading control. (n=3 independent experiments).

FIG. 2C. Spectrophotometric fluorescence intensity measurement of FITC-Tf transcytosis from the apical side to the basolateral side of HBMECs transfected with control siRNA or siRNA targeting STX-4. (n=3 independent experiments). This figure shows that silencing STX-4 by siRNA decreased Tf transcytosis by 3.30-fold (30.3% vs 100%) in in vitro Transwell assay (Spectrophotometric fluorescence intensity data 1066 vs 3516).

FIGS. 2D and 2E. Colocalization of STX-4 (red) with TfR (green) in polarized HBMECs (FIG. 2D), cell nuclei were stained with DAPI (blue). Scale bar, 2 μm. Quantification of colocalization was shown by calculating the Pearson correlation coefficients (PC), Mander's coefficient M1 (fraction of STX-4 colocalized with TfR) and M2 (fraction of TfR colocalized with STX-4). n=10 random areas per group from three independent experiments. Polarized HBMECs transfected with the pGMILV-SC5 lentiviral vector harboring shRNA targeting VAMP3 or control shRNA (FIG. 2E).

FIG. 2F. Colocalization of STX-4 (red) with gp135 (white, top) or E-cadherin (white, bottom) in polarized HBMECs, respectively. Quantification of colocalization was shown by calculating the Pearson correlation coefficients (PC). n=10 random areas per group from three independent experiments. Scale bar, 2 μm.

FIG. 2G. Colocalization of E-cadherin with TfR in polarized HBMECs transfected with the pGMLV-SC5 lentiviral vector harboring shRNA targeting VAMP3, STX-4 or control shRNA. Quantification of colocalization was shown by calculating the Pearson correlation coefficients (PC). n=10 random areas per group from three independent experiments.

Data are shown as the mean±SD, *P<0.05, **P<0.01, ***P<0.001, ns, nonsignificant. Two-tailed unpaired Student's t test (FIG. 2A, FIG. 2C), Two-way ANOVA (FIG. 2E), One-way ANOVA (2G). This shows a decrease in the fusion of TfR vesicles with the basolateral membrane when VAMP3 or syntaxin 4 were silenced with siRNA.

FIGS. 3A-3H. Overexpression of VAMP3 and syntaxin 4 (STX-4) enhances the efficiency of TfR?transcytosis.

FIG. 3A. Colocalization of VAMP3 with TfR in polarized VAMP3-overexpressing (H-VAMP3) cells and control cells. Quantification of colocalization was shown by calculating the Pearson correlation coefficients (PC), Mander's coefficient M1 (fraction of VAMP3 colocalized with TfR) and M2 (fraction of TfR colocalized with VAMP3). n=10 random areas per group from three independent experiments.

FIG. 3B. Colocalization of E-cadherin with TfR in polarized VAMP3-overexpressing cells (H-VAMP3) and control cells. Quantification of colocalization was shown by calculating the Pearson correlation coefficients (PC), Mander's coefficient M1 (fraction of E-cadherin colocalized with TfR) and M2 (fraction of TfR colocalized with E-cadherin). n=10 random areas per group from three independent experiments.

FIG. 3C. Spectrophotometric fluorescence intensity measurement of FITC-Tf transcytosis from the apical side to the basolateral side of VAMP3-overexpressing (H-VAMP3) cells and control cells. This figure shows that stably overexpressing VAMP3 in HBMECs increased FITC-Tf transcytosis by 2.46-fold (246% vs 100%) in in vitro Transwell assay (Spectrophotometric fluorescence intensity data 6676 vs 2719).

FIG. 3D. Colocalization of STX-4 with TfR in polarized STX-4-overexpressing (H-STX-4) cells and control cells. Quantification of colocalization was shown by calculating the Pearson a correlation coefficients (PC), Mander's coefficient M1 (fraction of STX-4 colocalized with TfR) and M2 (fraction of TfR colocalized with STX-4). n=10 random areas per group from three independent experiments.

FIG. 3E. Colocalization of E-cadherin with TfR in polarized STX-4-overexpressing (H-STX-4) cells and control cells. Quantification of colocalization was shown by calculating the Pearson correlation coefficients (PC), Mander's coefficient M1 (fraction of E-cadherin colocalized with TfR) and M2 (fraction of TfR colocalized with E-cadherin). n=10 random areas per group from three independent experiments.

FIG. 3F. Spectrophotometric fluorescence intensity measurement of FITC-Tf transcytosis from the apical side to the basolateral side of STX-4-overexpressing (H-STX-4) cells and control cells. This figure shows that stably overexpressing STX-4 in HBMECs increased FITC-Tf transcytosis by 2.02-fold (202% vs 100%) in in vitro Transwell assay (Spectrophotometric fluorescence intensity data 6017 vs 2983).

FIGS. 3G and 3H. Spectrophotometric fluorescence intensity measurement of FITC-Tf transcytosis from the apical side to the basolateral side of control cells compared with VAMP3-overexpressing (H-VAMP3) cells (FIG. 3G) or STX-4-overexpressing (H-STX-4) cells (FIG. 3H) transfected with control siRNA and STX-4 siRNA (3G), or VAMP3 siRNA (3H). n=3 independent experiments.

Data are shown as the mean±SD, *P<0.05, **P<0.01, ***P<0.001, ns, nonsignificant. Two-way ANOVA (FIGS. 3A, 3B, 3D, 3E, 3G, 3H), Two-tailed unpaired Student's t test (FIGS. 3C, 3F).

FIGS. 4A-4J. VAMP3 and syntaxin 4 (STX-4) contribute to the transcytosis of NMEC.

FIGS. 4A and 4B. Bacterial transcytosis in infected HBMECs transfected with control siRNA, VAMP1, VAMP2, VAMP3, VAMP4, VAMP7, or VAMP8 siRNA (4A) or STX-4 siRNA (4B). FIG. 4A shows that silencing VAMP3 by siRNA decreased NMEC transcytosis by 3-fold (0.009% vs 0.027%) in in vitro Transwell assay. FIG. 4B shows that silencing STX-4 by siRNA decreased NMEC transcytosis by 5.5-fold (0.004% vs 0.022%) in in vitro Transwell assay.

FIGS. 4C and 4D. Colocalization of intracellular NMEC with VAMP3 in infected HBMECs (FIG. 4C) or HBMECs transfected with control siRNA or RalA siRNA (FIG. 4D). The numbers indicate the percentage of BCVs colocalized with VAMP3 relative to total intracellular BCVs (n=3 slides). Bar=5 μm. Ral-A refers to a Ras-related protein.

FIG. 4E. Colocalization of VAMP3 with TfR in infected HBMECs transfected with control siRNA or RalA siRNA. Quantification of colocalization was shown by calculating the Pearson correlation coefficients (PC). n=10 random areas per group from three independent experiments.

FIG. 4F. Colocalization of STX-4 (red) with the intracellular NMEC (green) in polarized HBMECs. Bar=5 μm FIG. 4G. Colocalization of intracellular NMEC with STX-4 in polarized HBMECs transfected with the pGMILV-SC5 lentiviral vector harboring shRNA targeting VAMP3 or control shRNA. The numbers indicate the percentage of BCVs colocalized with STX-4 relative to total intracellular BCVs (n=3 slides).

FIG. 4H. Colocalization of STX-4 (red), VAMP3 (white) and intracellular NMEC (green) in polarized HBMECs. Bar=5 μm.

FIG. 4I. Colocalization of intracellular NMEC with E-cadherin in polarized HBMECs transfected with the pGMLV-SC5 lentiviral vector harboring shRNA targeting VAMP3, STX-4 or control shRNA. The numbers indicate the percentage of BCVs colocalized with E-cadherin relative to total intracellular BCVs (n=3 slides).

FIG. 4J. The effect of VAMP3 on penetration of the BBB by NMEC in vivo. Eighteen-day-old C57BL/6N mice and VAMP3^{-/-} mice (n=20) were infected with NMEC via the tail vein and sacrificed at 4 h p.i. The blood samples were collected for bacteremia measurement and the CSF were collected and cultured to indicate the incidence of meningitis. The positive CSF cultures were defined as meningitis. This figure shows that NMEC transcytosis across the BBB (animals with positive cerebrospinal fluid) was undetected in VAMP3^{-/-} mice in vivo.

Data are shown as the mean±SD, *P<0.05, **P<0.01, ***P<0.001, ns, not significant. One-way ANOVA (FIGS. 4A, 4I), Two-tailed unpaired Student's t test (4B, 4D, 4E, 4G).

FIGS. 5A to 5P. NMEC increases its transcytosis efficiency by enhancing the expression of VAMP3 and syntaxin 4 (STX-4).

FIGS. 5A and 5C. qRT-PCR analysis of VAMP3 (5A) or STX-4 (5C) expression in HBMECs and HBMECs infected by NMEC.

FIGS. 5B and 5D. Representative western blotting image and quantitative analysis of VAMP3 (FIG. 5B) or STX-4 (FIG. 5D) in HBMECs and HBMECs infected by NMEC. GAPDH, loading control (FIG. 5B). Hsp70, loading control (FIG. 5D).

FIGS. 5E and 5F. qRT-PCR analysis of VAMP3 (FIG. 5E) or STX-4 (FIG. 5F) expression in HBMECs and HBMECs treated by LPS.

FIGS. 5G and 5H. qRT-PCR analysis of VAMP3 or STX-4 expression in HBMECs and HBMECs infected by NMEC WT, ΔmsbB or complemented strain ΔmsbB+. Data represent the mean±SD (n=3).

FIG. 5I shows that stably overexpressing VAMP3 in HBMECs increased NMEC transcytosis by 3.79-fold (0.091% vs 0.024% vs) in in vitro Transwell assay.

FIG. 5J shows that stably overexpressing STX-4 in HBMECs increased NMEC transcytosis by 3.23-fold (0.084% vs 0.026%) in in vitro Transwell assay.

FIG. 5K describes NMEC transcytosis (% bacterial load) using si-VAMP3 control vs. H-STX4.

FIG. 5L describes NMEC transcytosis (% bacterial load) using si-STX4 control vs. H-VAMP3.

FIG. 5M describes colocalization rate of NMEC and VAMP3(%) in H-VAMP3 and control cells.

FIG. 5N describes colocalization rate of NMEC and STX4(%) in H-STX4 and control cells.

FIG. 5O describes colocalization rate of NMEC and E-cadherin (%) in H-VAMP3 and control cells.

FIG. 5P describes colocalization rate of NMEC and E-cadherin (%) in H-STX4 and control cells.

FIGS. 6A-6K. The expression of VAMP3 and syntaxin 4 (STX-4) expression is induced via TLR4-TRIF-dependent signaling pathway.

FIGS. 6A to 6F. qRT-PCR analysis of VAMP3 (FIGS. 6A, 6C, 6E) and STX-4 (FIGS. 6B, 6D, 6F) expression in HBMECs transfected control siRNA or siRNA targeting TLR4 (FIGS. 6A, 6B), TRAM, TRIF, TRAF3, IKK, IRF3 (FIGS. 6C, 6D), TIRAP, NF-κB (FIGS. 6E, 6F) in response to NMEC infection.

FIG. 6G. Bacterial transcytosis in infected HBMECs transfected with control siRNA or siRNA targeting TIRAP or NFκB.

FIG. 6H. Colocalization of intracellular NMEC with TfR in HBMECs transfected with control siRNA or siRNA targeting TLR4, TRAM, TRIF, TRAF3, IKK, IRF3.

FIG. 6I. Bacterial transcytosis in infected HBMECs transfected with control siRNA or IRF3 siRNA.

FIGS. 6J and 6K. Spectrophotometric fluorescence intensity measurement of FITC-Tf transcytosis from the apical side to the basolateral side of the HBMECs transfected with control siRNA or siRNA targeting TRAM, TRIF, TRAF3, IKK, IRF3 (FIG. 6J), TIRAP or NFκB (FIG. 6K) in response to LPS treatment.

Data are presented as the means±SD (n=3). *P<0.05; **P<0.01; ***P<0.001; ns represents no significance. Two-way ANOVA (FIGS. 6A-6F), One-way ANOVA (FIGS. 6G, 6H, 6J, 6K), Two-tailed unpaired Student's t test (FIG. 6I).

FIGS. 7A-7C. IRF3 directly regulates the expression of VAMP3.

FIG. 7A. Surface plasmon resonance assays using Biacore X100 SPR showed that 6×His-tagged IRF3 interacts with the promoter region of VAMP3, but not the syntaxin 4 promoter FIG. 7B. IRF3 binds to a motif in the promoter region of VAMP3. The protected region shows a significantly reduced peak intensities (blue) pattern than seen in compared with those of the control (red). The identified IRF3-binding motif is shown in a box at the bottom of the figure.

SEQ ID NO: 9 describes the sequence.

FIG. 7C. ChIP-qPCR analysis of the enrichment of the promoter region of VAMP3. IgG samples served as negative control.

Data are presented as the mean±SD (n=3). *P<0.05; **P<0.01; ***P<0.001; ns represents no significance. Two-tailed unpaired Student's t test (7C).

FIGS. 8A-8F. HBMECs were transfected with siRNA targeting VAMP1 (FIG. 8A), VAMP2 (FIG. 8B), VAMP3 (FIG. 8C), VAMP4 (FIG. 8D), VAMP7 (FIG. 8E), VAMP8 (FIG. 8F), or with control siRNA. The silencing efficiency is indicated at the top of the blots. β-actin, loading control. (n=3). Silencing of VAMP1, VAMP2, VAMP4, VAMP7 or VAMP8 by siRNA (reduction by 90-96%, FIG. 8C) did not affect the transcytosis of FITC-Tf across the HBMEC monolayer as shown by FIG. 1A.

FIG. 9. HBMECs were transfected with siRNA targeting STX-4, or with control siRNA.

The silencing efficiency is indicated at the top of the blots. Hsp70, loading control. (n=3).

Silencing syntaxin 4 (STX-4) shows a reduction by 97%. A significantly reduced amount of FITC-Tf transcytosed from the apical side (facing the lumen) to the basolateral side of polarized HBMEC monolayer transfected with siRNA targeting STX-4 in Transwells (FIG. 2C).

FIGS. 10A-10B. Colocalization of STX-4 (green) with E-cadherin (red, top, FIG. 10A) or gp135 (red, lower, FIG. 10B) in polarized STX-4-overexpressing (H-STX-4) cells, respectively, revealed by confocal microscopy analysis. Scale bar, 5 μm. The result indicates that STX-4 is only present at the basolateral membrane of H-STX-4. Confocal microcopy showed that in HBMECs stably overexpressing syntaxin 4 which was also only present at the basolateral membrane.

FIGS. 11A-11D. VAMP3 and STX-4 contribute to the transcytosis of NMEC.

FIG. 11A. Bacterial invasion of HBMECs transfected with VAMP3 siRNA, STX-4 siRNA or control siRNA. (n=3). The results indicate that VAMP3 and STX-4 do not influence the NMEC invasion of HBMECs.

FIG. 11B. Colocalization of intracellular NMEC with TfR in polarized HBMECs transfected with VAMP3 siRNA, syntaxin4 siRNA or control siRNA, revealed by confocal microscopy analysis. The numbers indicate the percentage of BCVs colocalized with TfR relative to total intracellular BCVs (n=3 slides). The result indicates that VAMP3 and STX-4 do not influence the fusion of BCVs with TfR vesicles within HBMECs FIG. 11C. HBMECs were transfected with siRNA targeting RalA, or with control siRNA.

The silencing efficiency is indicated at the top of the blots. β-actin, loading control. (n=3).

FIG. 11D. Colocalization of intracellular NMEC with VAMP3 in HBMECs pretreated with g/mL cycloheximide, revealed by confocal microscopy analysis. The numbers indicate the percentage of BCVs colocalized with TfR relative to total intracellular BCVs (n=3 slides). The result indicates that inhibition of protein synthesis in HBMECs prior to NMEC infection did not influence the colocalization of VAMP3 with BCVs. Data are shown as the mean±SD, *P<0.05, **P<0.01, ***P<0.001, ns, nonsignificant. One-way ANOVA (A, B), Two-tailed unpaired Student's t test (D). VAMP3 or syntaxin 4 had no effect on the bacterial invasion of HBMECs (FIG. 11A) and the fusion of BCVs with TfR vesicles within HBMECs (FIG. 11B). Silencing RalA (reduction by 93%, FIG. 11C), significantly reduced the colocalization of VAMP3 with BCVs (FIG. 4D). Inhibition of protein synthesis in HBMECs prior to NMEC infection had no effect on the colocalization of VAMP3 with BCVs (FIG. 11D).

FIGS. 12A-12D. NMEC increases its transcytosis efficiency by enhancing the expression of VAMP3 and STX-4. FIGS. 12A, 12C: qRT-PCR analysis of VAMP8 (FIG. 12A) or STX5 (FIG. 12C) expression in HBMECs and HBMECs infected by NMEC. The result indicates that NMEC infection does not influence the expression of VAMP8 and STX5 in HBMECs. (FIGS. 12B, 12D) Representative western blotting image and quantitative analysis of VAMP8 FIG. 12B) or STX5 (FIG. 12D) in HBMECs and HBMECs infected by NMEC. GAPDH, loading control (12B). Hsp70, loading control (FIG. 12D). The result confirms that NMEC infection does not influence the production of VAMP8 and STX5 in HBMECs.

As controls, the expression of VAMP8 and syntaxin 5, which are also present on the BCVs in HBMECs, exhibited no significant change in response to NMEC infection (FIGS. 12A-12D).

Treatment of HBMECs by LPS results in the up regulation of VAMP3 and syntaxin 4 (FIGS. 5E and 5F). Data represent the mean±SD (n=3). Two-tailed unpaired Student's t test.

FIGS. 13A-13H. HBMECs were transfected with siRNA targeting TLR4, TRAM, TRIF, TRAF3, IKK, IRF3, TIRAP, NF-xB or with control siRNA. The silencing efficiency is indicated at the top of the blots. β-actin, loading control. (n=3). Upregulation of VAMP3 and syntaxin 4 expression in response to NMEC infection was not influenced when TIRAP or NFκB was silenced a reduction by 87-95%, FIGS. 13G and 13H. See FIGS. 6E and 6F.

### DETAILED DESCRIPTION OF THE INVENTION

The challenge of delivering therapeutics into the central nervous system (CNS) has led to the exploration of various receptor-mediated transcytosis (RMT) methods in drug development. RMT is a mechanism by which drugs or therapeutic agents are transported across the blood-brain barrier (BBB) via specific receptors that facilitate the movement of molecules through endothelial cells. This strategy holds significant potential for the delivery of large or complex molecules, such as proteins, antibodies, or conjugated drugs, across the BBB to treat CNS conditions, including neurodegenerative diseases, brain cancer, amyloidosis, and other neurological disorders that benefit from targeted CNS drug concentrations. It can be particularly advantageous for achieving focused delivery while minimizing systemic side effects.

This disclosure focuses on the discovery of two novel proteins that have not previously been described for RMT, specifically in relation to transferrin receptor (TfR) vesicle binding. This discovery enhances the efficiency of drug-bound transcytosis (drug delivery) into the CNS. The two proteins are designed to exploit the BBB by binding to TfR receptors on human brain microvascular endothelial cells (HBMECs). Thus, increased expressions of and/or enhanced interactions between these two proteins holds promise to improve the efficiency of an antibody or ligand carrying a CNS-targeted large molecule or biologic to cross BBB. As part of the vesicular transport process, once the vesicle is transported across the endothelial cell, it exits the opposite membrane, enabling the delivery of the therapeutic agent to the CNS.

The inventors have found that three major meningitis-causing bacterial pathogens with different evolutionary history, including neonatal meningitis Escherichia coli (NMEC), Streptococcus pneumoniae and group B Streptococcus, which invade the brain from the bloodstream to cause diseases, have employed the same mechanism to cross the BBB. They all activate the same fusion process of bacteria-containing vesicles (BCVs) with TfR vesicles within HBMECs, thereby hijacking TfR transcytosis to penetrate the BBB. Cheng Z, et al. (2023) Pathogenic bacteria exploit transferrin receptor transcytosis to penetrate the blood-brain barrier. Proc Natl Acad Sci USA 120(39):e2307899120. This finding further demonstrates that utilizing TfR transcytosis for the transport of drugs across the BBB may be a promising option.

For TfR transcytosis across the BBB, holo-Tf (Tf-Fe) first binds to the TfR at the apical membrane (blood side) of HBMECs, and the resulting Tf-TfR complex enters the cell as vesicles through clathrin-mediated endocytosis, forming TfR vesicles. McCarthy R C & Kosman D J (2015) Mechanisms and regulation of iron trafficking across the capillary endothelial cells of the blood-brain barrier. Front Mol Neurosci 8:31. For approximately 90% of the endocytosed Tf-TfR complex, the iron is released from Tf in cytoplasm, and this portion of the Tf-TfR complex does not reach the basolateral membrane of HBMECs. McCarthy R C & Kosman D J (2015) Mechanisms and regulation of iron trafficking across the capillary endothelial cells of the blood-brain barrier. Front Mol Neurosci 8:31. The rest of the endocytosed holo-Tf traffics to the basolateral membrane (brain side) of HBMECs. These vesicles then fuse with the basolateral membrane of HBMECs, which is critical for the ultimate release of holo-Tf into the brain. Preston J E, Joan Abbott N, & Begley D J (2014) Transcytosis of macromolecules at the blood-brain barrier. Adv Pharmacol 71:147-163. However, the mechanism governing this process remains unclear. The inventors hypothesized that increasing the fusion of TfR vesicles with basolateral membrane of HBMECs, based on understanding the mechanism underlying this membrane fusion process, can enhance the efficiency of TfR transcytosis across the BBB.

SNARE proteins, divided into v-SNAREs on vesicles and t-SNAREs on target membranes, are the principal elements responsible for membrane fusion in different cells {Chen, 2001 #5000}. In non-polarized CHO cells and HeLa cells, the v-SNARE protein VAMP2 or VAMP3 present on TfR vesicles have been found to mediate the fusion of TfR vesicles with plasma membrane that lacks distinct polarity, respectively, contributing to the exocytic event of recycling TfR vesicles. Kubo K, et al. (2015) SNAP23/25 and VAMP2 mediate exocytic event of transferrin receptor-containing recycling vesicles. Biol Open 4(7):910-920; Galli T, et al. (1994) Tetanus toxin-mediated cleavage of cellubrevin impairs exocytosis of transferrin receptor-containing vesicles in CHO cells. J. Cell Biol. 125(5):1015-1024; Daro E, van der Sluijs P, Galli T, & Mellman I (1996) Rab4 and cellubrevin define different early endosome populations on the pathway of transferrin receptor recycling. Proc Natl Acad Sci USA 93(18):9559-9564. It remains unclear which SNARE proteins in polarized HBMECs are involved in the fusion between TfR vesicles and the basolateral membrane.

In this disclosure the inventors reveal that VAMP3 present on TfR vesicles and TfR-NMEC vesicles interacts with syntaxin 4 at the basolateral membrane of HBMECs, mediating the transcytosis of Tf and NMEC. Silence or mutation of VAMP3 and syntaxin 4 reduced the transcytosis of Tf and NMEC across the BBB in vitro and in mice. The inventors discovered that NMEC infection induces VAMP3 and syntaxin 4 expression via the activation of TLR4-TRAM-TRIF-TRAF3-IKK-IRF3 regulatory pathway by LPS. Furthermore, the inventors showed that high overexpression of VAMP3 or syntaxin 4 promote the fusion of TfR vesicles and TfR-NMEC vesicles with the basolateral membrane of HBMECs, and thus increasing the efficiency of TfR mediated transcytosis. This new work provides essential insights for developing strategies for the treatment of meningitis caused by NMEC and improvement of drug delivery into the brain.

### Definitions

Blood brain barrier (“BBB”). The blood-brain barrier (BBB) is a highly selective semipermeable border that separates the circulating blood from the brain and extracellular fluid in the central nervous system. It is formed by specialized endothelial cells lining the cerebral microvessels, known as Human Brain Microvascular Endothelial Cells (HBMECs).

Human Brain Microvascular Endothelial Cells (HBMECs) play a crucial role in maintaining the blood-brain barrier (BBB) and facilitating iron transport to the brain through the production of transferrin receptor (TfR) vesicles. HBMECs express high levels of transferrin receptors on their luminal (blood-facing) surface, which is essential for the uptake of iron-bound transferrin from the bloodstream. Upon binding of iron-loaded transferrin to TfR, clathrin-mediated endocytosis is triggered, leading to the formation of TfR-containing vesicles within the cell. These TfR vesicles undergo intracellular trafficking, moving through early endosomes where the acidic environment facilitates the release of iron from transferrin. A portion of these vesicles is directed towards the abluminal (brain-facing) side of the HBMECs, enabling the process of transcytosis. This transcytosis allows for the transport of iron across the BBB, ensuring its delivery to the brain. After releasing their cargo, the TfR vesicles can be recycled back to the luminal surface, allowing for continuous cycles of iron uptake and transport. The production and trafficking of TfR vesicles in HBMECs are tightly regulated, influenced by factors such as iron levels, cellular energy status, and various signaling pathways. This ability of HBMECs to produce and traffic TfR vesicles is critical for maintaining iron homeostasis in the brain, which is essential for numerous neurological functions. The efficient functioning of this mechanism underscores its importance in the overall maintenance of the BBB and neurological health. In the context of the present disclosure, these TfR vesicles may be fused or loaded with cells, proteins, drugs, or other agents and transcytosed through HBMECs and the BBB into the brain.

Stages of Transcytosis and the Roles of VAMP3 and Syntaxin-4. Transcytosis is a cellular process that transports macromolecules across the interior of a cell, typically involving three main stages: endocytosis, vesicular transport, and exocytosis.

Endocytosis is the initial stage where macromolecules are captured in vesicles formed from the plasma membrane. This stage involves receptor-mediated endocytosis: Specific receptors on the cell surface bind to the macromolecules, triggering the invagination of the plasma membrane and the formation of endocytic vesicles. Clathrin-Mediated Endocytosis: Often involves clathrin-coated pits that facilitate the internalization of the vesicles.

VAMP3 is primarily involved in the later stages of endocytosis, particularly in the recycling of endosomes. It helps in the sorting and trafficking of endocytic vesicles to their appropriate intracellular destinations. Syntaxin-4 is less involved in the initial endocytosis process but plays a role in the fusion of vesicles with target membranes during the later stages of transcytosis.

Vesicular Transport. Once inside the cell, the endocytic vesicles are transported across the cell to the opposite membrane. This involves vesicle sorting: The vesicles are sorted in early endosomes and directed towards recycling endosomes or other intracellular compartments.

Vesicles are transported along the cytoskeleton (microtubules and actin filaments) using motor proteins.

VAMP3 is a key player in vesicular transport, particularly in the recycling endosome pathway. It is involved in the trafficking of vesicles from the Golgi apparatus to the plasma membrane and from endosomes to the plasma membrane. Syntaxin-4 is involved in the targeting and docking of vesicles at specific membrane sites. It forms part of the SNARE complex that mediates the fusion of vesicles with the target membrane, ensuring the correct delivery of cargo Exocytosis is the final stage where the vesicles fuse with the plasma membrane on the opposite side of the cell, releasing their contents into the extracellular space. This stage involves vesicle docking where vesicles are docked at the plasma membrane and membrane fusion where the vesicle membrane fuses with the plasma membrane, facilitated by SNARE proteins.

VAMP3 is a v-SNARE protein that play a crucial role in the membrane fusion process during exocytosis. It pairs with t-SNAREs (such as syntaxin-4 and SNAP-23) on the target membrane to form the SNARE complex, driving the fusion of vesicles with the plasma membrane Syntaxin-4 is a t-SNARE protein located on the plasma membrane. It interacts with VAMP3 and SNAP-23 to form the SNARE complex, which is essential for the docking and fusion of vesicles during exocytosis. Syntaxin-4 is particularly important in activity-dependent exocytosis, such as the release of neurotransmitters and other signaling molecules.

Within the context of the invention, the expression or concentration of VAMP3 and STX-4 molecules may be modulated to control transcytosis. Reduction of the expression or reduced quantities of these molecules in vesicles can reduce transcytosis and tighten the BBB to permeation of molecules, for example, to prevent entry of harmful molecules or cells. In contrast, increased expression or increased concentrations of these molecules in vesicles can enhance transcytosis.

Vesicle-associated membrane proteins are a family of SNARE proteins involved in vesicle fusion. VAMPs are small integral membrane proteins found in secretory vesicles. There are several types of VAMP proteins, including: VAMP1 and VAMP2: Expressed primarily in the brain and found in synaptic vesicles. VAMP3 (cellubrevin): Ubiquitously expressed and involved in regulated and constitutive exocytosis. VAMP4: Involved in transport from the Golgi apparatus. VAMP5: Found in secretory vesicles, myotubes, and tubulovesicular structures. VAMP7: Present in both secretory granules and endosomes. VAMP8 (endobrevin): Participates in endocytosis and regulated exocytosis in pancreatic acinar cells. Modulation of VAMP3 is disclosed herein.

Soluble N-ethylmaleimide-sensitive factor Attachment protein Receptor (SNARE). The Soluble N-ethylmaleimide-sensitive factor Attachment protein Receptor (SNARE) complex is a group of proteins that mediate the fusion of vesicles with their target membranes in eukaryotic cells. This complex consists of v-SNAREs (vesicle SNAREs) and t-SNAREs (target SNAREs) that interact to facilitate membrane fusion, which is crucial for processes such as exocytosis, endocytosis, and intracellular transport. VAMP3 (Vesicle-associated membrane protein 3) is classified as a v-SNARE because it is localized on the vesicular membranes. It partners with t-SNARE proteins such as syntaxin-4 (STX-4), which is located on the target membranes, to form the SNARE complex during the fusion process. Therefore, both VAMP3 and STX-4 are SNARE proteins, with VAMP3 serving as the v-SNARE and STX-4 acting as the t-SNARE. This interaction between VAMP3 and STX-4 is essential for the precise and efficient fusion of vesicles with their target membranes, facilitating various cellular transport and signaling processes. Modulation of expression, concentrations of, and interaction of SNARE proteins VAMP3 and STX-4 are disclosed herein.

Neonatal meningitis-causing Escherichia coli (NMEC) is a distinct pathotype of extraintestinal pathogenic E. coli (ExPEC) that is a significant cause of bacterial meningitis in newborns. There is diversity among neonatal meningitis Escherichia coli (NMEC) strains, but some key characteristics distinguish them from other types of E. coli.

Serogroups: While many NMEC strains are untypeable, the most common serogroups include: O18 (most prevalent), O83, O7, O12, O1, O75 and O2.

Phylogenetic group: NMEC strains are more likely to belong to the B2 phylogenetic group compared to fecal E. coli from healthy individuals.

Sequence types: The most common sequence types among NMEC are ST95 and ST1193.

Virulence factors: NMEC strains are more likely to possess certain virulence genes, including fimbrial adhesins, iron acquisition systems, K1 capsule, genes from coli genomic and plasmid pathogenicity islands.

Specific genes: Some genes that distinguish NMEC from fecal E. coli include sfa/foc (S fimbriae, ibeA (invasion of brain endothelium), neuC (K1 capsule synthesis), sitA (iron transport), and vat (vacuolating autotransporter toxin).

Antimicrobial resistance: NMEC strains may show low levels of antimicrobial resistance to particular antibiotics compared to other E. coli pathotypes such as commensal E. coli. NMEC strains generally show high sensitivity to several antibiotics, including gentamicin, imipenem, meropenem, piperacillin/tazobactam, and amikacin. However, they can exhibit resistance to commonly used antibiotics like ampicillin and tetracycline. Commensal E. coli strains also show high sensitivity to many antibiotics but may have lower overall resistance due to fewer virulence factors and resistance genes.

Components of NMECs. Outer Membrane Proteins (OMPs):NMEC's outer membrane protein A (OmpA) interacts with host cell receptors, potentially triggering signaling cascades that could upregulate VAMP3 expression. OmpA has been shown to bind to the Ecgp96 receptor on brain endothelial cells, which may initiate cellular responses affecting vesicle trafficking proteins like VAMP3.

Lipopolysaccharide (LPS):LPS, a major component of the NMEC cell wall, is known to stimulate inflammatory responses. This could lead to increased expression of various proteins involved in vesicle trafficking, including VAMP3, as part of the cellular response to infection.

Type 1 Fimbriae:NMEC strains often express type 1 fimbriae, which are involved in bacterial adhesion to host cells. The interaction of these fimbriae with host cell receptors may trigger signaling pathways that upregulate VAMP3 as part of the cellular response to bacterial attachment.

Cytotoxic Necrotizing Factor 1 (CNF1): Some NMEC strains produce CNF1, which can modulate host cell signaling and cytoskeleton rearrangement. These changes could potentially lead to increased VAMP3 expression as part of the cellular adaptation to bacterial invasion.

Iron Acquisition Systems: NMEC strains often possess multiple iron acquisition systems, which are crucial for their survival in the host. The activation of these systems during infection may indirectly influence VAMP3 expression through alterations in cellular iron homeostasis and related signaling pathways.

K1 Capsule: The K1 capsule, a common virulence factor in NMEC, helps the bacteria evade the host immune response. This evasion may lead to prolonged bacterial presence, potentially resulting in sustained cellular responses that could include upregulation of VAMP3.

Inflammatory Mediators: While not a direct component of NMEC, the inflammatory response triggered by the bacteria (including cytokine production) could lead to increased VAMP3 expression as part of the overall cellular response to infection.

One or more of these components may be used in conjunction with the invention, for example, to be incorporated into vesicles that later fuse with HBMECs or otherwise increase or modulate transcytosis.

TLR4-TRAM-TRIF-TRAF3-IKK-IRF3 regulatory pathway: the TLR4-TRAM-TRIF-TRAF3-IKK-IRF3 regulatory pathway is a crucial signaling cascade in the innate immune system, particularly in response to bacterial lipopolysaccharide (LPS) and other pathogen-associated molecular patterns (PAMPs).

Syntaxin 4 (STX-4) function and location: Syntaxin 4 is a plasma membrane t-SNARE protein involved in vesicle trafficking and exocytosis. It is expressed in various tissues, including brain, pancreatic beta cells, and adipocytes In neurons, STX-4 is localized post-synaptically and regulates trafficking of proteins to the postsynaptic membrane.

E-cadherin (Cadherin-1): Cell-adhesion protein encoded by the CDH1 gene. E-cadherin is a key component of adherens junctions (protein complexes that allow cells to adhere to each other), structures that facilitate strong cell-cell adhesion in epithelial tissues. It helps maintain tissue architecture and polarity, which is crucial for normal tissue function and homeostasis.

VAMP3 sequences. Representative VAMP3 sequences include those polynucleotide sequence described by and incorporated by reference to NCBI Reference Sequence: NM_004781.4 (Homo sapiens)(SEQ ID NO: 1) and the amino acid sequence described by NCBI Preference Sequence. NP_004772.1 (Homo sapiens)(SEQ ID NO: 2) as described at www.ncbi.nlm.nih.gov/nuccore/NM_004781.4 or by Gene ID: 9341, updated on 7 Jul. 2024; www.ncbi.nlm.nih.gov/gene/9341. Source, gene, exon, misc_feature, CDS, poly A site and other information disclosed by these reference sequences as of Jul. 24, 2024 is expressly incorporated by reference to these reference sequences.

Syntaxin-4 [STX-4] sequences. Representative STX-4 sequences include those polynucleotide sequence described by and incorporated by reference to NCBI Reference Sequence NM_00127209.1 (Homo sapiens)(SEQ ID NO: 3) and the amino acid sequence described by NCBI Reference Sequence: “NP_001259024.1 (Homo sapiens)(SEQ ID NO: 4) as described at www.ncbi.nlm.nih.gov/nuccore/NM_001272095.1 or by Gene ID: 6810, updated on 17 Jun. 2024; www.ncbi.nlm.nih.gov/gene/6810. Source, gene, exon, misc_feature, CDS, poly A site and other information disclosed by these reference sequences as of Jul. 24, 2024 is expressly incorporated by reference to these reference sequences.

Variants may include, but are not limited to, fragments of the full-length sequence, polypeptides or polynucleotides having one or more amino acid or nucleotide substitutions, insertions, or deletions, and chemically modified versions thereof. Fragments may comprise at least 10, 20, 50, 100, or more contiguous amino acids or nucleotides of the reference sequence, while maintaining substantial biological activity. Substitution variants may possess at least 80%, 90%, 95%, or 99% sequence identity to the reference sequence. Chemical modifications may include, but are not limited to, pegylation, glycosylation, phosphorylation, or the incorporation of non-natural amino acids or nucleotides, designed to enhance stability, half-life, or biological activity in vivo. These variants are contemplated to retain substantial biological function of the reference sequence while potentially offering improved pharmacokinetic properties, reduced immunogenicity, enhanced stability, or optimized activity profiles for therapeutic or diagnostic applications.

A variant sequence, such as a VAMP3 or STX-4 variant or mutant, includes sequences with one or more deletions, substitutions or additions of polynucleotides or amino acid residues, for example, to the sequences given by SEQ ID NOS: 1 and 2; or for STX-4 the sequences given by SEQ ID NOS: 3 and 4. It includes fragments of a longer molecule, such as fragments comprising a known active site or domain or encoding a known active site or domain. A variant or mutant polynucleotide sequence or polypeptide sequence may have 1, 2, 3, 4, 5, 6, 7, 8, 9, 10 or more deletions, substitutions or insertions to a known sequence of another polynucleotide or amino acid residue.

Variants may be described by reference to a particular degree of sequence identity to a known molecule, such as to a polynucleotide encoding the VAMP3 amino acid sequence described by SEQ ID NOS: 1-4. For example, a polynucleotide encoding VAMP3 may have at least 70, 75, 80, 85, 90, 95, 99 or <100 sequence identity to a known polynucleotide; and a variant VAMP3 polypeptide may have at least 70, 75, 80, 85, 90, 95, 99 or <100 sequence identity to a known VAMP 3 amino acid sequence like SEQ ID NO: 2.

BLASTN may be used to identify a polynucleotide sequence having at least 70%, 75%, 80%, 85%, 87.5%, 90%, 92.5%, 95%, 97.5%, 98%, 99% or <100% sequence identity to a reference polynucleotide such as a polynucleotide encoding VAMP3 or encoding a siRNA binding to a VAMP3 transcript. A representative BLASTN setting modified to find highly similar sequences uses an Expect Threshold of 10 and a Wordsize of 28, max matches in query range of 0, match/mismatch scores of 1/−2, and linear gap cost. Low complexity regions may be filtered or masked. Default settings of a Standard Nucleotide BLAST are described by and incorporated by reference to hypertext transfer protocol secure://blast.ncbi.nlm.nih.gov/Blast.cgi?PROGRAM=blastn&PAGE_TYPE=BlastSearch&LINK_LOC=blasthome (last accessed Jul. 24, 2024).

BLASTP can be used to identify an amino acid sequence having at least 70%, 75%, 80%, 85%, 87.5%, 90%, 92.5%, 95%, 97.5%, 98%, 99% or <100% sequence identity, or similarity to a reference amino acid, such as a known VAMP3 amino acid sequence, using a similarity matrix such as BLOSUM45, BLOSUM62 or BLOSUM80 where BLOSUM45 can be used for closely related sequences, BLOSUM62 for midrange sequences, and BLOSUM80 for more distantly related sequences. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity or similarity to the amino acid sequences disclosed herein are contemplated and encompassed by this disclosure. A representative BLASTP setting that uses an Expect Threshold of 10, a Word Size of 3, BLOSUM 62 as a matrix, and Gap Penalty of 11 (Existence) and 1 (Extension) and a conditional compositional score matrix adjustment. These default settings for BLASTP are described by and incorporated by reference to the disclosure available at: hypertext transfer protocol secure://blast.ncbi.nlm.nih.gov/Blast.cgi?PROGRAM=blastp&PAGE_TYPE=BlastSearch&LIN K_LOC=blasthome (last accessed Jul. 24, 2024).

Mouse FLAG antibodies. Murine polyclonal or monoclonal antibodies that recognize DYKDDDDK (SEQ ID NO: 6).

Making siRNA. Small interfering RNA may be commercially produced to targeted mRNA sites, such as mRNA encoding VAMP3 or STX-4, as those described by and incorporated by reference to: www.thermofisher.com/us/en/home/references/ambion-tech-support/rnai-sirna/general-articles/-sirna-design-guidelines.html (last accessed Jul. 24, 2024) or produced by methods known in the art and incorporated by reference to Elbashir, et al. (2001) Functional anatomy of siRNA for mediating efficient RNAi in Drosophila melanogaster embryo lysate. EMBO J 20: 6877-6888; or Sui, G. et al. (2002) A DNA vector-based RNAi technology to suppress gene expression in mammalian cells. Proc. Natl. Acad. Sci. US A 99(8): 5515-5520. In some embodiments siRNA may be encapsulated in lipid nanoparticles to protect them from degradation and to facilitate delivery into target cells; may be incorporated into stable complexes comprising polyethyleneimine and cyclodextrin; or administered as conjugates with carriers that target the brain or other specific tissues. These may include conjugation to polymeric, lipid-based or gold nanoparticles, conjugation to exosomes, peptide conjugation, for example, to a cell-penetrating peptide, conjugation to an antibody targeting the brain or other organs, or expression by or conjugation to components of a vector or virus.

Methods for Enhancing VAMP3- and/or STX-4-mediated activity and VAMP3 and/or STX-4-mediated-transcytosis. To enhance VAMP3-mediated or STX-4-medicated transcytosis for drug delivery to the brain, several strategies and potential therapeutic agents can be considered.

Gene therapy using adenoviral vectors expressing VAMP3 or STX-4 can significantly increase its expression in endothelial cells. For instance, the intraluminal application of adenovirus expressing VAMP3 (Ad-VAMP3) has demonstrated the potential to enhance VAMP3 levels in endothelial cells, thereby improving vesicle trafficking and transcytosis.

Identifying small molecules that selectively activate pathways to enhance VAMP3 expression or function could be beneficial. Although mTORCl inhibitors like rapamycin are known to reduce VAMP3 expression, further research is required to discover small molecule activators that can upregulate VAMP3 effectively.

Designing peptides that enhance the formation of the SNARE complex involving VAMP3, syntaxin, and SNAP-23 can improve vesicle fusion and transcytosis. These peptides can mimic or stabilize the interactions within the SNARE complex, facilitating more efficient vesicle trafficking. Certain cytokines and growth factors can upregulate VAMP3 expression. For example, vascular endothelial growth factor (VEGF) and other signaling molecules involved in endothelial cell function have the potential to enhance VAMP3-mediated transcytosis.

Inhibiting miRNAs that negatively regulate VAMP3 expression can be a viable strategy to enhance its levels. For instance, miR-124 is known to downregulate VAMP3; therefore, using antagomirs or miRNA sponges to inhibit miR-124 can result in increased VAMP3 expression.

Designing nanoparticles that specifically target VAMP3 can improve drug delivery across the blood-brain barrier (BBB). These nanoparticles can be functionalized with ligands or antibodies that bind to VAMP3, facilitating their transport via VAMP3-mediated transcytosis.

Combining the aforementioned approaches can provide synergistic effects, enhancing the overall efficacy of drug delivery to the brain. For example, a combination of gene therapy and peptides that enhance VAMP3-mediated or STX-4-mediated transcytosis may be used. These may utilize adenoviral vectors to upregulate VAMP3 expression in conjunction with peptides that stabilize the SNARE complex can maximize the enhancement of transcytosis. A combination of small molecules and nanoparticles may also be considered stabilize or enhance VAMP3 or STX-4 roles in promoting transcytosis. Stabilizing VAMP3 or STX-4 with small molecules while using VAMP3-targeted nanoparticles can improve both the stability and delivery efficiency of therapeutic agents. These strategies collectively aim to enhance VAMP3 and/or STX-4 expression and function, thereby facilitating more efficient drug delivery to the brain through VAMP3-mediated transcytosis.

Modes of administration. The administration of microRNA (miRNA) or other therapeutics to the brain can be achieved through various modes, each designed to overcome the blood-brain barrier (BBB) and enhance delivery to target tissues. Direct administration methods include intracerebroventricular (i.c.v.) injection into the brain ventricles, intrathecal catheter (ITC) delivery to the cisterna magna, and intrastriatal (i.s.) injection for region-specific targeting. Nanoparticle-mediated delivery systems, such as lipid-based, polymeric, and gold nanoparticles, as well as exosomes, can be engineered to encapsulate miRNA and facilitate BBB penetration.

Receptor-mediated transcytosis utilizing transferrin or rabies viral glycoprotein (RVG) tags enhances BBB crossing. Intranasal administration offers a non-invasive approach for nose-to-brain delivery, bypassing the BBB. Additionally, molecular Trojan horses like pegylated immunoliposomes (PILs) conjugated with targeting antibodies can deliver miRNA across the BBB. Novel carriers such as argininocalixarene-based systems and magnetic nanoparticles (e.g., Neuromag®) show promise for improved miRNA delivery to the brain. These diverse administration modes aim to optimize miRNA stability, cellular uptake, and targeted delivery for potential therapeutic applications in neurological disorders.

Target molecules such as pharmaceutic drugs or biologicals may be chemically conjugated to transferrin, metal-based drugs may be complexed with iron-binding sites on transferrin, a target molecule may be encapsulated in nanoparticles that are conjugated to transferrin, a fusion protein between peptide-based biological and transferrin may be used, or bifunctional or multifunctional linkers may be used to connect a target molecule to transferrin or its receptor-binding residues. In some alternative embodiments cellular receptors other than transferrin receptors, such as insulin, LDL, LRP1, and neonatal Fc receptors, as well as those described below may be used to transport a target molecule. Similar modifications to a target molecule may be made when using these non-transferrin receptors.

The following methods and compositions may be used in conjunction with the methods for modulating VAMP3 and/or Syntaxin 4 (STX-4) levels or expression.

Transferrin Receptor Binding (TfR) with Antibody Drug Conjugates (ADC):

Transferrin Receptor (TfR)-Binding Antibody Drug Conjugates (ADCs) are an advanced method being developed to deliver therapeutic agents across the blood-brain barrier (BBB) for treating central nervous system (CNS) diseases.
This approach leverages the process of receptor-mediated transcytosis, which is utilized by molecules like transferrin (a key iron-transporting protein) to cross the BBB.
TfR-binding ADCs represent a promising approach for the development of CNS therapies, especially for diseases where delivery of large molecules or biologics is needed.
These advancements could revolutionize treatment options for brain diseases by providing more efficient and targeted drug delivery methods across the blood-brain barrier.

Advantages of TfR-Binding ADCs include:
Enhanced Brain Delivery: These conjugates take advantage of an already existing and efficient transport mechanism, improving the delivery of therapeutics to the brain.
Specificity: Using antibodies that specifically bind to the transferrin receptor minimizes off-target effects, reducing systemic toxicity.
Wide Applicability: This approach can be adapted to deliver a variety of therapeutic agents, from small molecules to large biologics, making it versatile for treating numerous CNS disorders, including Alzheimer's disease, brain tumors, and neurodegenerative diseases.

### Mechanism of TfR-Binding Antibody Drug Conjugates

The blood-brain barrier is a highly selective barrier that limits the entry of most therapeutic molecules into the brain, presenting a major challenge for CNS drug development. TfR-binding ADCs use the transferrin receptor to transport drugs across the BBB.
TfR as a Gateway: The transferrin receptor (TfR) is expressed on the endothelial cells of the BBB. It is responsible for transporting transferrin-bound iron from the blood into the brain. By exploiting this receptor, therapeutics can cross the barrier that would otherwise block them.
Conjugating Therapeutics to Antibodies: The drugs or therapeutic agents (such as small molecules, peptides, or other biologics) are linked to an antibody or ligand that binds specifically to the TfR. The most common approach uses monoclonal antibodies (mAbs) that recognize the TfR and bind to it with high affinity.
Receptor-Mediated Transcytosis: Once the TfR-targeting ADC binds to the transferrin receptor on the surface of endothelial cells, the receptor undergoes endocytosis, forming an intracellular vesicle. The vesicle carries the complex across the endothelial cell and releases the therapeutic agent on the brain side of the BBB.
Drug Release in the Brain: Once inside the brain, the drug is released from the antibody, either via intracellular mechanisms (such as enzymatic cleavage) or through controlled release from the drug conjugate. The released drug then exerts its therapeutic effect on the targeted brain cells.

Examples of TfR-related drugs which may be used in conjunction with the compositions and methods disclosed herein are shown in the Table A below.

TABLE A
Examples of TfR related drugs in clinical development

| Generic Name | Trade name | Indication | Mechanism |
|---|---|---|---|
| JR-141 1 | CoraFluor ™ | Hunter syndrome | JR-141 is a fusion protein that |
| In Phase 3 | (Company: JCR | (Mucopolysaccharidosis | combines the enzyme (iduronate-2- |
| | Pharmaceuticals), | Type II) | sulfatase, which is deficient in |
| | | | patients with Hunter syndrome) with |
| | | | an antibody against the TfR. It utilizes |
| | | | RMT mechanism to cross the BBB, |
| | | | allowing the therapeutic agent to |
| | | | reach CNS. |
| DNL310 | (Tividenofusp | Hunter syndrome | DNL310 utilizes Denali Therapeutics' |
| In Phase 2/3 | Alfa ™) | (Mucopolysaccharidosis | Transport Vehicle (TV) platform, |
| | (Company: Denali | Type II) | which involves conjugating the |
| | Therapeutics) | | deficient enzyme (iduronate-2- |
| | | | sulfatase) to a ligand that binds to the |
| | | | TfR. This binding allows the drug to |
| | | | be transported across the BBB via |
| | | | transcytosis, delivering the enzyme to |
| | | | the CNS. |
| RO7126209 | (Trontinemab ™) | Alzheimer's disease | The anti-amyloid monoclonal |
| In Phase 1/2 | (Company: Roche) | | antibody gantenerumab's effector (Fc |
| | | | domain) conjugated with a Fab |
| | | | fragment that binds the human |
| | | | transferrin receptor. The drug utilizes |
| | | | RMT via the TfR to transport the anti- |
| | | | amyloid beta antibody into the CNS. |
| | | | By binding to the TfR on endothelial |
| | | | cells of the BBB, RO7126209 can be |
| | | | carried across this barrier and |
| | | | delivered into the brain, where it |
| | | | targets and clears amyloid-beta |
| | | | plaques associated with Alzheimer's |
| | | | disease. |

Other RMT Techniques which May be Used in Conjunction with the Methods and Compositions Disclosed Herein which Modulate VAMP3 and/or STX-4 Expression or Levels Include:
1. Insulin Receptor-Targeting Peptides
   Mechanism: Therapeutic agent, e.g. iduronate-2-sulfatase, which is an enzyme lacking in Hunter syndrome, is linked to peptides or proteins that bind to insulin receptors, which are abundantly expressed on endothelial cells of the BBB. Upon binding, the complex undergoes transcytosis, delivering the drug to treat Hunter Syndrome.
2. Monoclonal Antibodies (mAbs) for Neurodegenerative Diseases
   Example: PBT2, an experimental drug for Alzheimer's disease and Huntington's disease, is being investigated using RMT pathways. The drug binds to low-density lipoprotein receptors (LDLRs), utilizing the receptor-mediated pathway to cross the BBB.
   Mechanism: Monoclonal antibodies or fragments conjugated with ligands for LDLR-related proteins (LRP-1 and LRP-2) are used to ferry the therapeutic across the BBB. Once inside, they reduce toxic beta-amyloid and tau aggregates in the brain.
3. Angiopep-2-Linked Chemotherapeutics
   Example: ANG1005 (paclitaxel-derivative conjugated with Angiopep-2)
Mechanism: Angiopep-2 binds to the low-density lipoprotein receptor-related protein (LRP-1), which is overexpressed in the BBB and various brain tumors. By exploiting this pathway, ANG1005 effectively crosses the BBB to deliver chemotherapy to brain tumors like glioblastoma, improving the efficacy of treatment.
4. P97 Shuttle Peptide Conjugates
   Mechanism: P97, also known as melanotransferrin, naturally crosses the BBB via receptor-mediated transcytosis by interacting with transferrin receptors. Conjugating drugs to P97 allows targeted delivery into the CNS. This strategy is being explored for neurodegenerative diseases, including Parkinson's disease and ALS.
5. Lipid Nanoparticle and Nanocarrier Systems
   Lipid nanoparticles (LNPs) and other nanocarrier systems are increasingly used to ferry larger therapeutic molecules across the BBB.
6. Examples of drugs in development using RMT techniques:
   BIIB080 (Antisense Oligonucleotide)—Company: Biogen
      Indication: Alzheimer's disease.
      Mechanism: Antisense oligonucleotide targeting tau protein mRNA to reduce tau aggregation in Alzheimer's disease.
      BBB Strategy: Leveraging insulin receptor-mediated transcytosis or through direct administration into the CNS via intrathecal injection.
   ANG1005—Company: Angiochem
      Indication: Glioblastoma and brain metastases.
      Mechanism: Paclitaxel conjugated to Angiopep-2 peptide, which targets LRP1 (low-density lipoprotein receptor-related protein 1) on the BBB.
      BBB Strategy: Angiopep-2 enables paclitaxel to cross the BBB using LRP1-mediated transcytosis, enhancing chemotherapy delivery to brain tumors.
   DNL201 and DNL151 (LRRK2 inhibitors)—Company: Denali Therapeutics
      Indication: Parkinson's disease.
      Mechanism: Small-molecule inhibitors targeting LRRK2 (Leucine-rich repeat kinase 2) mutations associated with Parkinson's disease.
      BBB Strategy: Denali's proprietary “Transport Vehicle (TV)” platform uses receptor-mediated transcytosis via LRP1 to enhance CNS penetration.

TABLE B
List of FDA approved drugs or compounds that target CNS and need to cross the BBB.

| Generic Name | Trade Name | Indication | Mechanism |
|---|---|---|---|
| Bevacizumab | Avastin ™(Genentech/Roche) | Glioblastoma | VEGF (vascular endothelial |
| | | | growth factor) inhibitor |
| | | | (mAb) that reduces blood |
| | | | vessel formation in tumors, |
| | | | allowing better delivery of |
| | | | chemotherapy to brain |
| | | | tumors. Systemic delivery |
| | | | without specific BBB |
| | | | targeting but effective in |
| | | | tumors with leaky |
| | | | vasculature, may benefit from |
| | | | RMT. |
| Cerezyme | Imiglucerase ™ (Genzyme) | Gaucher disease | Enzyme Replacement |
| | | | Therapies (ERTs) - Although |
| | | | FDA approved for Gaucher |
| | | | disease, the systemic drug |
| | | | cannot cross the BBB |
| | | | effectively and highlights the |
| | | | challenge of BBB penetration |
| | | | for enzyme replacement |
| | | | therapies in CNS-related |
| | | | manifestations of lysosomal |
| | | | storage diseases. |
| Levodopa/Carbidopa | Sinemet ™ ™ | Parkinson's disease | Levodopa is a dopamine |
| | | | precursor, crossing the |
| | | | BBB and is converted |
| | | | into dopamine to increase |
| | | | levels in the brain |
| | | | Carbidopa prevents |
| | | | peripheral conversion of |
| | | | levodopa to dopamine, |
| | | | allowing more to reach |
| | | | the CNS |
| Memantine | Namenda ™ | Alzheimer's disease | NMDA receptor antagonist, |
| | | | modulates glutamate activity |
| Escitalopram | Lexapro ™ | Depression and | Selective serotonin reuptake |
| | | anxiety disorders | inhibitor (SSRI), increases |
| | | | serotonin levels in the brain |
| Duloxetine | Cymbalta ™ | Major depressive | Serotonin-norepinephrine |
| | | disorder, | reuptake inhibitor (SNRI), |
| | | generalized anxiety | boosts serotonin and |
| | | disorder, and | norepinephrine |
| | | neuropathic pain | |
| Rivastigmine | Exelon ™ | Alzheimer's and | Acetylcholinesterase |
| | | Parkinson's-related | inhibitor, increases |
| | | dementia | acetylcholine |
| Gabapentin | Neurontin | Epilepsy, | Modulates GABA |
| | | neuropathic pain | neurotransmission |
| Riluzole | Rilutek ™ | Amyotrophic lateral | Inhibits glutamate release and |
| | | sclerosis (ALS) | enhances glutamate reuptake, |
| | | | protecting motor neurons |
| | | | from excitotoxic damage |
| Fingolimod | Gilenya ™ | Multiple sclerosis | Sphingosine-1-phosphate |
| | | (MS) | receptor modulator, reduces |
| | | | lymphocyte migration into |
| | | | the CNS |
| Naltrexone | ReVia ™ | Alcohol and opioid | Opioid receptor antagonist, |
| | | dependence | modulates CNS opioid |
| | | | receptors |
| Phenytoin | Dilantin ™ | Epilepsy | Blocks voltage-gated sodium |
| | | | channels in neurons, |
| | | | preventing repetitive firing |
| | | | and stabilizing neuronal |
| | | | activity to reduce seizures |
| Temozolomide | Temodar ™ | Glioblastoma | Alkylating agent, which |
| | | | damages DNA in cancer |
| | | | cells, leading to cell death and |
| | | | tumor regression |
| Ocrelizumab | Ocrevus ™ | Multiple Sclerosis | Anti-CD20 monoclonal |
| | | (MS) | antibody, which targets B |
| | | | cells and reduces their ability |
| | | | to attack myelin in the CNS, |
| | | | slowing MS progression |
| Carbamazepine | Tegretol ™ | Epilepsy, bipolar | Sodium channel blocker, |
| | | disorder | which stabilizes hyperactive |
| | | | neurons to reduce seizures |
| | | | and mood swings |
| Baclofen | Lioresal ™ | Muscle spasticity in | GABA-B receptor agonist, |
| | | multiple sclerosis | which inhibits excitatory |
| | | and spinal cord | neurotransmission in the |
| | | injury | CNS and reduces muscle |
| | | | spasticity |
| Lamotrigine | Lamictal ™ | Epilepsy, bipolar | Inhibits sodium channels and |
| | | disorder | stabilizes glutamate release, |
| | | | reducing seizures and mood |
| | | | fluctuations |
| Clozapine | Clozaril ™ | Treatment-resistant | Dopamine D2 and serotonin |
| | | schizophrenia | 5-HT2 receptor antagonist, |
| | | | with lower affinity for D2 |
| | | | receptors, which treats |
| | | | psychotic symptoms with |
| | | | reduced risk of |
| | | | extrapyramidal side effects |
| Dexamethasone | Decadron ™ | Cerebral edema | Corticosteroid, which |
| | | (swelling in the | reduces inflammation and |
| | | brain), brain tumors | edema by suppressing the |
| | | | immune response |
| Lorazepam | Ativan ™ | Anxiety disorders, | Benzodiazepine that |
| | | epilepsy | enhances GABA activity, |
| | | | leading to sedative, |
| | | | anxiolytic, and |
| | | | anticonvulsant effects |
| Sumatriptan | Imitrex ™ | Migraine | Serotonin (5-HT1) receptor |
| | | | agonist, which constricts |
| | | | cranial blood vessels and |
| | | | inhibits neuropeptide release, |
| | | | relieving migraine pain |
| Zonisamide | Zonegran ™ | Epilepsy | Sodium and calcium channel |
| | | | blocker, which reduces |
| | | | neuronal excitability and |
| | | | prevents seizures |
| Methylphenidate | Ritalin ™, Concerta ™ | Attention | Blocks dopamine and |
| | | Deficit | norepinephrine reuptake, |
| | | Hyperactivity | increasing their levels in the |
| | | Disorder (ADHD) | CNS to improve attention and |
| | | | focus |

TABLE C
Investigational drugs in Clinical Development that target CNS and need to cross the BBB.

| Generic Name | Trade name | Indication | Mechanism |
|---|---|---|---|
| AGT-182 | (Company: | Hunter Syndrome | Fusion of iduronate-2-sulfatase (an |
| | ArmaGen | (Mucopolysaccharidosis | enzyme lacking in Hunter syndrome) |
| | Technologies) | II) | with an insulin receptor antibody. |
| | | | BBB Strategy: Receptor-mediated |
| | | | transcytosis via insulin receptor to |
| | | | deliver enzyme replacement therapy |
| | | | across the BBB. |
| P97 Shuttle System | (Company: Bioasis | Alzheimer's, | P97, a melanotransferrin-derived |
| | Technologies) | Parkinson's, and brain | peptide, binds to TfR for transcytosis |
| | | cancer | across the BBB. BBB Strategy: Uses |
| | | | the P97 shuttle to deliver biologics, |
| | | | including mAbs and enzymes |
| Lecanemab | | Alzheimer's disease | Anti-amyloid beta antibody, aims to |
| | | | reduce amyloid plaques in the brain |
| Tofersen | | Amyotrophic lateral | Antisense oligonucleotide (ASO) |
| | | sclerosis (ALS) with | targeting mutant SOD1 mRNA |
| | | SOD1 mutation | |
| BIIB080 | | Alzheimer's disease | Antisense oligonucleotide targeting |
| | | | tau protein production |
| Trofinetide | | Rett syndrome | Neurotrophic factor analog, aims to |
| | | | improve synaptic function |
| Caplyta | Lumateperon ™ e | Schizophrenia and | Dopamine D2 receptor modulator and |
| | | bipolar disorder | serotonin receptor antagonist |
| Simufilam | | Alzheimer's disease | Restores normal shape and function |
| | | | of altered filamin A in the brain |
| Niraparib | | Brain metastases in | PARP inhibitor, aimed at DNA repair |
| | | certain cancers | pathways in brain tumors |
| Tavapadon | | Parkinson's disease | Partial dopamine D1/D5 receptor |
| | | | agonist |
| Anavex 2-73 | Blarcamesine ™ | Alzheimer's disease | Sigma-1 receptor agonist, modulates |
| | | and Rett syndrome | protein folding and neuroprotection |
| Masitinib | | ALS and Alzheimer's | Tyrosine kinase inhibitor targeting |
| | | disease | microglial activation in the CNS |
| LY3002813 | | Alzheimer's disease | Anti-amyloid beta antibody, targets |
| | | | amyloid plaques |
| Reldesemtiv | | ALS | Fast skeletal muscle troponin |
| | | | activator, improves muscle function |
| | | | by enhancing calcium sensitivity |
| BIIB054 | Cinpanemab ™ | Parkinson's disease | Anti-alpha-synuclein antibody, |
| | | | targets abnormal protein aggregates |
| ANG1005 | | Glioblastoma | Paclitaxel-derivative conjugated with |
| | | | Angiopep-2 |
| Tocagen | Toca 511 ™ & Toca | Glioblastoma -newly | A two-part therapy where Toca 511, a |
| | FC ™ | diagnosed cases | retroviral replicating vector, delivers |
| | | | a gene into tumor cells, making them |
| | | | susceptible to Toca FC, an orally |
| | | | administered antifungal drug that |
| | | | converts into a toxic agent inside the |
| | | | cancer cells |
| Onc-201 | Imipridone ™ | Glioblastoma - | Small-molecule drug that selectively |
| | | recurrent (H3 K27M- | targets dopamine receptor D2 |
| | | mutant glioma) | (DRD2), leading to apoptosis in |
| | | | cancer cells |
| ABT-414 | Depatuxizumab ™ | Glioblastoma - newly | Antibody-drug conjugate targeting |
| | Mafodotin ™ | diagnosed and | the EGFR (epidermal growth factor |
| | | recurrent | receptor), which is often |
| | | | overexpressed in glioblastoma cells |
| DNX-2401 | Delta-24-RGD ™ | Glioblastoma - | Oncolytic adenovirus designed to |
| | | recurrent | selectively replicate in and destroy |
| | | | cancer cells, while sparing normal |
| | | | cells |
| VBI-1901 | | Glioblastoma - | Cancer vaccine that targets |
| | | recurrent | cytomegalovirus antigens expressed |
| | | | in glioblastoma cells |
| VAL-083 | Dianhydro- | Glioblastoma - | Alkylating agent that induces |
| | galactitol ™ 1 | resistant to | interstrand DNA cross-links, leading |
| | | temozolomide | to cell death |
| Marizomib | | Glioblastoma - used | Proteasome inhibitor that disrupts |
| | | in combination with | protein degradation pathways, |
| | | temozolomide and | leading to cancer cell death |
| | | radiation | |
| SurVaxM | | Glioblastoma | Peptide vaccine that targets survivin, |
| | | | a protein that inhibits cancer cell |
| | | | death and is overexpressed in |
| | | | glioblastoma |
| Rindopepimut | | Glioblastoma | Vaccine targeting the EGFRvIII |
| (CDX-110) | | | mutation, commonly present in |
| | | | glioblastoma cells |
| PBT2 | | Alzheimer's disease | Reduces toxic beta-amyloid |
| | | and Huntington's | and tau aggregates |
| | | disease | |

### Methods to Increase VAMP3 and STX-4 Protein Expression

As described in the manuscript and patent draft, bacterial LPS can induce the expression of VAMP3 and STX-4 in HBMECs via the TLR4-TRIF-dependent signaling pathway. However, this method will induce inflammation.

Adeno-associated virus-based (AAV) technology targeting BBB maybe used to enhance the expression of VAMP3 and syntaxin 4 in HBMECs, utilizing vector expressing VAMP3 and syntaxin 4; see. Körbelin J, et al. (2016) A brain microvasculature endothelial cell-specific viral vector with the potential to treat neurovascular and neurological diseases. EMBO Mol Med. 8(6):609-25 which is incorporated by reference.

Lipid nanoparticle based-delivery systems also have the potential to deliver VAMP3 and syntaxin 4 mRNA into BBB; see Anderson D M, et al. (2003) Stability of mRNA cationic lipid lipoplexes in human and rat cerebrospinal fluid: methods and evidence for nonviral mRNA gene delivery to the central nervous system. Hum Gene Ther 14(3):191-202 which is incorporated by reference.

The microRNA miR-124 represses VAMP3 expression in microglia making it a potential target for regulating VAMP3 expression. Enhancing VAMP3 expression maybe achieved by silencing miR-124; see (Chen Y, et al. (2019) miR-124 VAMP3 is a novel therapeutic target for mitigation of surgical trauma-induced microglial activation. Signal Transduct Target Ther 4:27), which is incorporated by reference.

In drug delivery and gene therapy, the strategy of increasing the expression of all genes in a cell is generally not used, due to the unforeseen side effects. However, the expression of VAMP3 and STX-4 may be targeted to brain endothelial cells using cell-specific promoter.

### Examples

### Materials & Methods

Bacterial strains. E. coli strain RS218, a prototype meningitis isolate from the CSF of a newborn infant, was designated as NMEC. Huang S H, et al. (1999) Identification and characterization of an Escherichia coli invasion gene locus, ibeB, required for penetration of brain microvascular endothelial cells. Infect. Immun. 67(5):2103-2109. Mutant ΔmsbB of NMEC was generated using the λ-Red recombination system. Datsenko K A & Wanner B L (2000) One-step inactivation of chromosomal genes in Escherichia coli K-12 using PCR products. Proc. Natl. Acad. Sci. USA 97(12):6640-6645. The complementary strain ΔmsbB+ was established by cloning msbB and native promoters into the pACYC-184 plasmid and transformed into NMEC. Bacterial strains were grown in Luria-Bertani (LB) broth.

Cell culture conditions and transfections. The HBMEC cell line was cultured in Dulbecco's Modified Eagle Medium (DMEM)(Gibco) supplemented with 10% fetal bovine serum (FBS)(Excell Bio), 10% Nu-Serum (BD Biosciences), 100 units/mL penicillin, 100 μg/mL streptomycin and incubated at 37° C. in 5% CO₂. To stably knock-down gene expression, shRNA targeting the specific gene or a scrambled control shRNA was designed and constructed into the lentiviral vector pGMLV-SC5 (Genomeditech). 293T cells were used for lentivirus packaging with HBMECs. To stably overexpress VAMP3 or syntaxin 4, VAMP3 overexpression plasmid were constructed in the GM-19315r by Genomeditech. Syntaxin 4 overexpression plasmid were constructed in the lenti-CMV-MCS-PGK-Puro by Genomeditech. 293T cells were used for lentivirus packaging with HBMECs. For silencing gene expression transiently, HBMECs were transfected with negative control siRNA or siRNA targeting specific gene using Lipofectamine RNAi MAX (Invitrogen) according to the manufacture instruction.

Animal model. All animal experiments were performed according to the standards set forth in the Guide for the Care and Use of Laboratory Animals. The experimental protocols were approved by the Institutional Animal Care Committee at Nankai University. 18-day-old C57BL/6J mice were purchased from Beijing Vital River Laboratory Animal Technology Co., Ltd. Gene-deficient VAMP3^{-/-} mice were purchased from Model Organisms Center (Shanghai, China), maintained on a C57BL/6J background.

NMEC invasion assays. Bacterial strains were grown overnight in LB broth at 37° C. and activated to the exponential phase at an optical density of 600 nm (OD_{600 nm}) of 0.6. Bacteria were pelleted, washed with PBS and resuspended in infection medium [medium 199 (Gibco) and Ham's F-12 (Gibco) (1:1) supplemented with 5% FBS]. After 90 min incubation of HBMECs with bacteria at a MOI of 100:1 and washing three times with PBS, HBMECs were incubated in DMEM medium containing 100 μg/mL gentamycin for 60 min to kill the extracellular bacteria. Then the cells were washed with sterile PBS for three times and lysed using 0.1% Triton X-100 in PBS. The bacteria were collected and enumerated by LB agar plates. Invasion was calculated as relative invasion compared to that for infection of WT HBMECs, defined as 100%. All assays were performed with at least three independent biological replicates.

Transwell assay. HBMECs were cultured on the inner surface of collagen-coated Transwell inserts (pore size 3.0 μm), containing 200 μL DMEM medium with 10% FBS and 10% Nu-Serum, and 1 mL DMEM medium with 10% FBS and 10% Nu-Serum was added in the lower chamber. HBMECs were seeded at a density of 2×10⁵cells/well in 200 μL medium onto 24-well Transwell chambers (Corning-Costar). Then, HBMECs were grown to confluency for at least 5 days. The integrity of the monolayers was monitored by measuring transendothelial electrical resistance (TEER) using ECIS TEER24 machine (Applied Biophysics Inc). No significant changes in TEER were found under all our experimental conditions, indicating that the polarized monolayers in the transwells were intact.

For FITC-Tf, permeability was measured by adding 0.1 mg/ml of FITC-Tf (Cat Number: T2871, Thermo Fisher Scientific) to the upper chamber, with the lower compartment containing fresh serum-free media. After incubation for 90 min, 100 μL of medium from the lower compartment was taken and measured for fluorescence at excitation 495 nm and emission 528 nm using a microplate reader (SpectraMax M5, Molecular Devices). All independent experiments were performed in triplicate.

For NMEC transcytosis, bacteria in 200 μL infection medium were added to the endothelial cell layers on Transwell filters at a MOI of 100:1 and incubated for 90 min. Both apical and basolateral chambers were washed with sterile PBS for three times and extracellular bacteria were killed by incubation in DMEM medium containing 100 μg/mL gentamycin for 60 min. After washed by sterile PBS for three times, both apical and basolateral chambers were replenished with DMEM medium with 25 μg/mL trimethoprim and 50 μM rottlerin, which prevents the reattachment and reentry of any expelled bacteria. After incubation for addition 4 hours at 37° C., 100 μL samples were taken from the apical and basolateral chambers respectively and enumerated by plating suitable dilutions on agar plates. In addition, HBMECs on the Transwell filter were lysed with 0.1% Triton X-100 and the samples were also collected and enumerated by plating suitable dilutions on agar plates. The percentage of bacterial number in the basolateral chamber to total bacterial count (all section of Transwell chambers containing apical, basolateral and filter) was described as bacterial transcytosis. All independent experiments were performed in triplicate.

Immunofluorescence microscopy. HBMECs were seeded on 20-mm diameter coverslips and cultured in DMEM medium at 37° C. Fixed with ice-cold 4% paraformaldehyde at 4° C. for 10 min, then permeabilized with 0.3% Triton for 20 min and blocked with 5% BSA at room temperature for 1 h. The fixed cells were incubated with primary antibodies diluted in 5% BSA at 4° C. overnight. To visualize with a confocal microscopy, the cells were washed with PBS for three times then incubated with respective fluorophore-conjugated secondary antibodies at room temperature for 1 h. Then the cells were washed three times with PBS and mounted in Prolong Gold Antifade Mountant with DAPI (Thermo Fisher Scientific).

When bacterial infection is included in the experiments, HBMECs were seeded on 20-mm diameter coverslips and cultured in DMEM medium at 37° C. for 24 h prior to infection. After 90 min incubation of HBMECs with NMEC at a MOI of 100:1, the HBMEC cells were treated as the above steps, stained with the appropriate antibodies and viewed by confocal microscopy.

For polarized HMBECs, the cells were grown on collagen filters, fixed in 4% paraformaldehyde at 4° C. for 10 min, permeabilized with 0.3% Triton for 20 min, and blocked with 5% BSA at room temperature for 1 h. Cells were stained with primary antibodies diluted in 5% BSA at 4° C. overnight and fluorophore conjugated secondary antibodies at room temperature for 1 h. After staining, Transwell membranes containing cells were excised with a scalpel and placed onto glass slides with glass coverslips and Prolong Gold Antifade Mountant with DAPI.

The primary antibody used to include anti-VAMP3 antibody (1:100 dilution), anti-STX-4 antibody (1:100 dilution), anti-TfR (1:100 dilution), anti-E-cadherin (1:100 dilution), anti-gp135 (1:100 dilution).

Three slides were examined for each sample in the above assay. 10 random areas or 100-150 BCVs were observed on each slide. Images were taken using a Zeiss LSM800 confocal microscope (Zeiss). The colocalization Mander's and Pearson's coefficients were analyzed using ImageJ.

Penetration of Biotin-Tf across the BBB of mice. Each mouse received Biotin-Tf via the tail vein. Zuchero Y J, et al. (2016) Discovery of Novel Blood-Brain Barrier Targets to Enhance Brain Uptake of Therapeutic Antibodies. Neuron 89(1):70-82. At 4 h after inoculation, CSF specimens were collected for quantitation of Biotin-Tf as described previously. Cheng Z, et al. (2023) Pathogenic bacteria exploit transferrin receptor transcytosis to penetrate the blood-brain barrier. Proc Natl Acad Sci USA 120(39):e2307899120. The level of Biotin-Tf were determined by Fluorescence Biotin Quantitation Kit (Thermo Fisher Scientific) according to the manufacture instruction.

In vivo bacterial penetration of the BBB. C57BL/6N wild type mice and gene-deficient VAMP3^{-/-} mice were used for the tail vein injection model. 1×10⁷ CFU of NMEC in 100 μL sterile PBS were injected intravenously in which doses bacteria develop a high level of bacteremia followed by bacterial traversal of the BBB mimicking the pathogenesis of human meningitis. Cheng Z, et al. (2023) Pathogenic bacteria exploit transferrin receptor transcytosis to penetrate the blood-brain barrier. Proc Natl Acad Sci USA 120(39):e2307899120. At 4 h after bacterial inoculation, blood and CSF specimens were collected for bacterial cultures. The bacterial numbers in the samples were determined by plating serial dilutions on LB agar.

Western blotting analysis. Proteins were separated in 4-12% SDS-PAGE and transferred to 0.22 m PVDF membranes (Millipore). Membranes were blotted with 5% non-fat milk in TBST (m/v) at 25° C. for 1 h, then incubated with indicated antibodies at 4° C. overnight, washing with TBST for three times and further incubated with HRP-conjugated goat anti-rabbit or -mouse IgG antibodies at 25° C. for 1 h. The primary antibody used includes: anti-VAMP3 antibody (1:1000 dilution), anti-STX-4 antibody (1:1000 dilution), anti-Hsp60 (1:1000 dilution), anti-GAPDH (1:1000 dilution), anti-Hsp70 (1:1000 dilution), anti-VAMP1 antibody (1:1000 dilution), anti-VAMP2 antibody (1:1000 dilution), anti-VAMP4 antibody (1:1000 dilution), anti-VAMP7 antibody (1:1000 dilution), anti-VAMP8 antibody (1:1000 dilution), anti-STX5 antibody (1:1000 dilution), anti-TLR4 antibody (1:1000 dilution), anti-TRAM antibody (1:1000 dilution), anti-TRIF antibody (1:1000 dilution), anti-TRAF3 antibody (1:1000 dilution), anti-IKK antibody (1:1000 dilution), anti-IRF3 antibody (1:1000 dilution), anti-TIRAP antibody (1:1000 dilution), anti-NF-κB antibody (1:1000 dilution). The bands were visualized with SuperSignal west pico chemiluminescent substrate (Thermo). Images were acquired using an Amersham™ Imager 600 System (General Electric Company). Protein levels were analyzed using ImageJ.

Co-immunoprecipitation (co-IP) analysis. Proteins were extracted from cultured HBMECs, and immunoprecipitation was performed using 2 μg polyclonal antibody against mouse FLAG (1:1,000, Beyotime Biotechnology). After 3 hr incubation, protein A magnetic bead (Thermo Fisher Scientific) was added and incubated overnight at 4° C., and then centrifuged for 1 min at 12,000 g. The precipitates were rinsed with immunoprecipitation buffer (0.5% NP-40, Tris-Cl pH8.0, 0.15 μM NaCl) four times to remove non-specific binding molecules. The protein levels in precipitates were then assessed by Western blotting.

Quantitative RT-PCR (qRT-PCR). Total RNA was extracted as previously described. RNA was treated with DNase I at 37° C. for 30 min and successful removal of DNA contamination. cDNA was synthesized using the PrimeScript™ RT reagent Kit (Takara; RR037A) according to the manufacturer's instructions. GAPDH was used as a reference to standardize expression across the samples. Samples were amplified and detected using SYBR green dye and an Applied Biosystems ABI 7500 sequence detection system (Applied Biosystems, CA, USA). The relative difference in gene expression was calculated using the cycle threshold method (2^{ΔΔct}). Data were collected from at least three biological replicates.

Dye primer-based DNase I footprinting assay. A 1000-bp fragment of the VAMP3 promoter regions was generated by PCR with 6-FAM-labeled primers: 5-CAATGACCTATTAGTGTTTTATTTT-3 (SEQ ID NO: 7) and 5-GGCGCGGCGCGGGGCAAA-3 (SEQ ID NO: 8). For IRF3 purification, the coding sequence of human IRF3 was cloned into pET32a plasmid and transformed into E. coli BL21 (DE3). The IRF3 with an N-terminal 6×His tag were expressed after subjection to induction using 0.5 mM IPTG for 6 h at 16° C. and purified by HiTrap Ni²⁺ chelating column (GE Healthcare). Protein concentrations were determined by the Bradford protein assay (Bio-Rad). Various amounts of 6×His-tagged IRF3 protein were added to 40 ng of 6-FAM-labeled VAMP3 promoter in a binding buffer (10 mM Tris-HCl [pH 7.5], 0.2 mM dithiothreitol, 5 mM MgCl₂, 10 mM KCl, and 10% glycerol). 0.05 U DNase I (Sigma, #AMPD1) was added to a 20 μL reaction for 15 min at 30° C. The reaction was stopped by heating at 70° C. for 10 min in the presence of 250 mM EDTA. DNA fragments were purified with the QIAquick PCR Purification Kit (Qiagen, #28104) and eluted in 15 μL distilled water. The samples were analyzed by MAP Biotech Co., Ltd. (Shanghai, China). The results were analyzed using a peak scanner (Applied Biosystems).

ChIP-qPCR. HBMEC cells were transfected with pcDNA3.1 overexpressing IRF3-FLAG or empty vector using Lipofectamine 3000 (Invitrogen) according to the manufacture instruction. The cells were collected at 48 h after transfection and formaldehyde was added to a final concentration of 1% (v/v) immediately. After incubated for 25 min, the cross-linking was quenched by the addition of 0.5 μM glycine. After washing for three times in ice-cold sterile PBS, the cells were resuspended in lysis buffer (50 mM Tris-HCl pH 7.5, 100 mM NaCl, 1 mM EDTA, 1 mM PMSF, 20 mg/mL lysozyme). After incubated at 37° C. for 30 min, the sample was sonicated to generate DNA fragments of approximately 250 to 500 bp. Insoluble cellular debris was removed via centrifugation for 20 min at 4° C., and the supernatant was collected for immunoprecipitation (IP) experiments as the input sample. The mock and IP samples were incubated with anti-mouse IgG and anti-FLAG antibodies (Sigma, #F1804), respectively, and then incubated with protein A magnetic beads (Invitrogen, #10002D) according to the manufacturer's instructions. The protein-DNA complexes were washed, reversed and purified with a PCR purification kit (Qiagen, #28104).

To measure the enrichment of potential IRF3-binding targets in the immunoprecipitated DNA samples, the percent of the input and fold enrichment were determined using SYBR green PCR master mix. Relative target levels were calculated using 2^{-ΔΔt} method, with GAPDH used as a negative control. The results are reported as the average enrichment of three independent biological replicates.

Surface plasmon resonance (SPR) assay. SPR measurements were performed using streptavidin-coated sensor chips (SA chip) on a Biacore™ X100 analytical system (GE Healthcare). SA-chip (Biacore™) was activated by injection of a mixture containing 50 mM NaOH and 1 M NaCl. The target nucleic acid VAMP3 and STX-4 were 3′-biotinylated and diluted to 100 μg/mL with deionised water, which keeping flow rate of 10 L/min 600 s and immobilised on the surface of the SA chip. Proteins were dialyzed overnight against HBS-EP+ buffer, and six different concentrations of each protein were prepared by serial dilution with HBS-T buffer for each set of sensorgrams.

The procedures for kinetic analyses were automated to perform repetitive cycles of sample injection and regeneration. Proteins were diluted in an isocratic gradient with HBS-EP+ buffer in a suitable concentration range.

The sample was analyzed at a flow rate of 30 l/min. The time of protein binding was 180 s and the time of dissociation was 300 s. At the end of each cycle, the chip surface was regenerated by injecting 0.5% SDS. The sensorgrams were processed for baseline alignment and reference channel subtraction with the Biacore™ X100 Evaluation Software (GE Healthcare). Kinetic analysis was performed by globally fitting the curves describing a simple 1:1 bimolecular model to the set of five sensorgrams.

Quantification and statistical analysis. Data were presented as the mean±SD. Statistical significance was analyzed with GraphPad Prism 9.3.1 software (GraphPad Inc., San Diego, CA) using the two-tailed unpaired Student's t test, one-way ANOVA, two-way ANOVA, or Mann-Whitney U-test according to the test requirements, as stated in the figure legends.

### Results

For TfR transcytosis across the BBB, holo-Tf (Tf-Fe) first binds to the TfR at the apical membrane (blood side) of HBMECs, and the resulting Tf-TfR complex enters the cell as vesicles through clathrin-mediated endocytosis, forming TfR vesicles. McCarthy R C & Kosman D J (2015) Mechanisms and regulation of iron trafficking across the capillary endothelial cells of the blood-brain barrier. Front Mol Neurosci 8:31. For approximately 90% of the endocytosed Tf-TfR complex, the iron is released from Tf in cytoplasm, and this portion of the Tf-TfR complex does not reach the basolateral membrane of HBMECs. McCarthy R C & Kosman D J (2015) Mechanisms and regulation of iron trafficking across the capillary endothelial cells of the blood-brain barrier. Front Mol Neurosci 8:31. The rest of the endocytosed holo-Tf traffics to the basolateral membrane (brain side) of HBMECs.

These vesicles then fuse with the basolateral membrane of HBMECs, which is critical for the ultimate release of holo-Tf into the brain. Preston J E, Joan Abbott N, & Begley D J (2014) Transcytosis of macromolecules at the blood-brain barrier. Adv Pharmacol 71:147-163. However, the mechanism governing this process remains unclear.

The inventors considered that increasing the fusion of TfR vesicles with basolateral membrane of HBMECs, based on understanding the mechanism underlying this membrane fusion process, can enhance the efficiency of TfR transcytosis across the BBB.

SNARE proteins, divided into v-SNAREs on vesicles and t-SNAREs on target membranes, are the principal elements responsible for membrane fusion in different cells. Chen Y A & Scheller R H (2001) SNARE-mediated membrane fusion. Nat Rev Mol Cell Biol 2(2):98-106. In non-polarized CHO cells and HeLa cells, the v-SNARE protein VAMP2 or VAMP3 present on TfR vesicles have been found to mediate the fusion of TfR vesicles with plasma membrane that lacks distinct polarity, respectively, contributing to the exocytic event of recycling TfR vesicles. Kubo K, et al. (2015) SNAP23/25 and VAMP2 mediate exocytic event of transferrin receptor-containing recycling vesicles. Biol Open 4(7):910-920; Galli T, et al. (1994) Tetanus toxin-mediated cleavage of cellubrevin impairs exocytosis of transferrin receptor-containing vesicles in CHO cells. J. Cell Biol. 125(5):1015-1024; Daro E, van der Sluijs P, Galli T, & Mellman I (1996) Rab4 and cellubrevin define different early endosome populations on the pathway of transferrin receptor recycling. Proc Natl Acad Sci USA 93(18):9559-9564. It remains unclear which SNARE proteins in polarized HBMECs are involved in the fusion between TfR vesicles and the basolateral membrane.

### Results

VAMP3 contributes to the transcytosis of Tf across the BBB. VAMP1, VAMP2, VAMP3, VAMP4, VAMP7 and VAMP8 are major v-SNARE proteins involved in the membrane fusion step of exocytosis process of different cells. Ireton K, Van Ngo H, & Bhalla M (2018) Interaction of microbial pathogens with host exocytic pathways. Cell Microbiol 20(8):e12861; Kubo K, et al. (2015) SNAP23/25 and VAMP2 mediate exocytic event of transferrin receptor-containing recycling vesicles. Biol Open 4(7):910-920; Hu C, Hardee D, & Minnear F (2007) Membrane fusion by VAMP3 and plasma membrane t-SNAREs. Exp Cell Res 313(15):3198-3209. The inventors first investigated whether some of these v-SNARE proteins are involved in the transcytosis of Tf through HBMECs, using a polarized HBMEC monolayer with tight junctions in Transwells in vitro as described previously. Cheng Z, et al. (2023) Pathogenic bacteria exploit transferrin receptor transcytosis to penetrate the blood-brain barrier. Proc Natl Acad Sci USA 120(39):e2307899120.

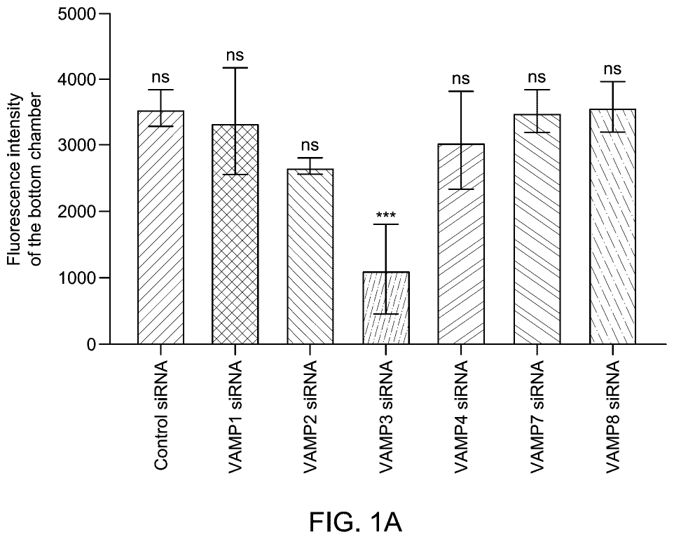

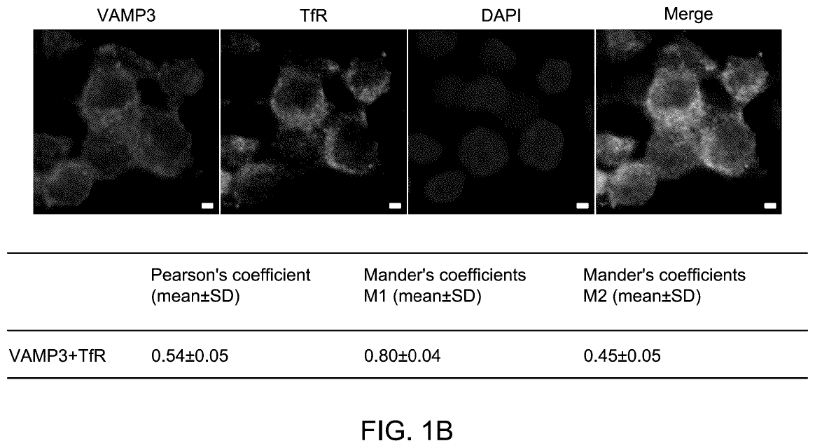

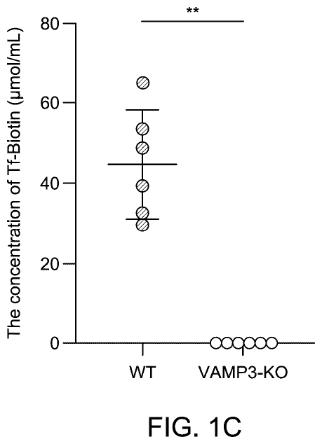

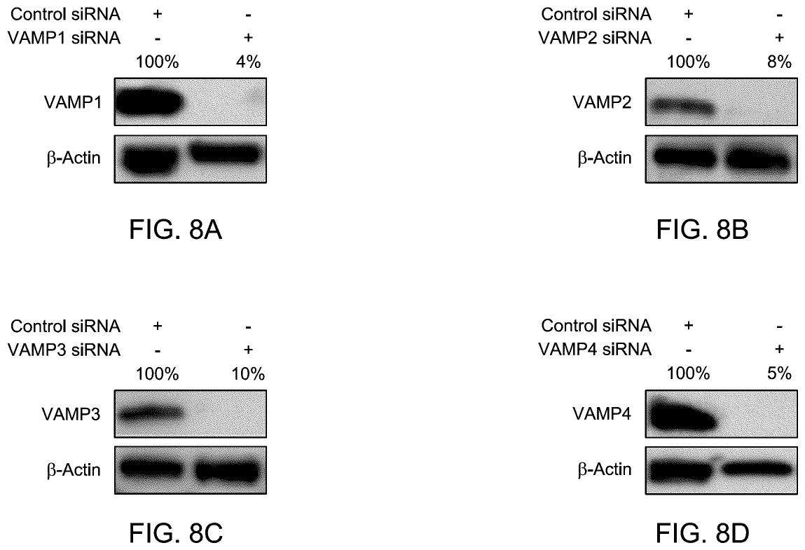

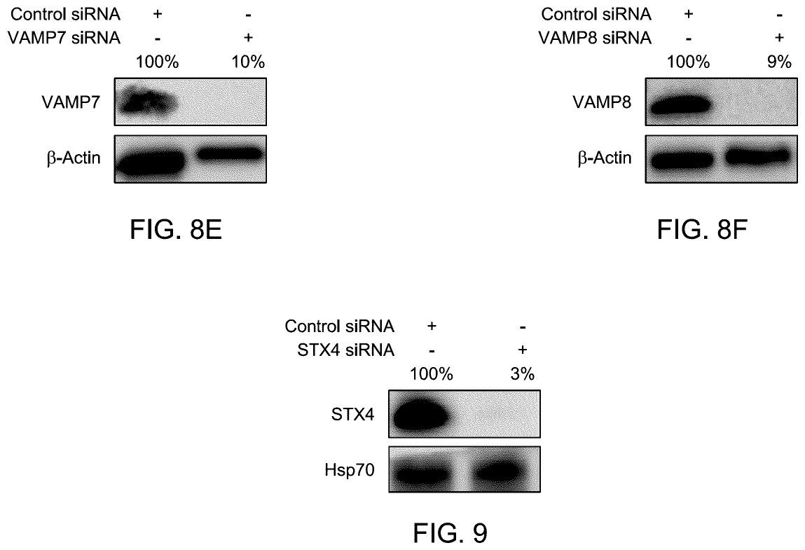

The inventors showed that silencing VAMP3 of HBMECs with siRNA (reduction by 90%, FIG. 8C) significantly reduced the amount of FITC-Tf transcytosed from the apical side to the basolateral side of the cell (FIG. 1A), however, silencing VAMP1, VAMP2, VAMP4, VAMP7 or VAMP8 by siRNA (reduction by 90-96%, FIGS. 8A, 8B, 8D, 8E, 8F and 8G) did not affect the transcytosis of FITC-Tf across the HBMEC monolayer (FIG. 1A).

Colocalization of VAMP3 with TfR was observed in HBMECs using confocal microscopy (FIG. 1). The Pearson's correlation and Manders' overlap coefficients were used to measure the colocalization of VAMP3 with TfR (Pearson's coefficient was 0.54; Manders' coefficient M1 (the fraction of VAMP3 colocalized with TfR) was 0.80, and M2 (the fraction of TfR colocalized with VAMP3) was 0.45). These data indicate that within HBMECs, the majority of VAMP3 is located on TfR vesicles, whereas a small fraction of TfR vesicles are positive for VAMP3.

Furthermore, the inventors investigated whether VAMP3 influences the penetration of Tf across the BBB in vivo. The inventors showed that gene-deficient VAMP3^{-/-} mice exhibited significantly reduced amount of FITC-Tf in the cerebrospinal fluid (CSF) after tail vein injection compared to wild-type mice (FIG. 1C), indicating the transcytosis of FITC-Tf across the BBB of VAMP3^{-/-} mice was significantly inhibited.

These data indicate that VAMP3 present on the TfR vesicles promotes the transcytosis of Tf across the BBB in vitro and in vivo.

The Interaction of VAMP3 with Syntaxin 4 at the Basolateral Membrane Mediates the Final Fusion Step of TfR Transcytosis Across HBMECs

VAMP3 has been reported to interact with t-SNARE syntaxin 4 to mediate the membrane fusion events in several different cells. Pulido I R, Jahn R, & Gerke V (2011) VAMP3 is associated with endothelial weibel-palade bodies and participates in their Ca(2+)-dependent exocytosis. Biochim. Biophys. Acta 1813(5):1038-1044; Hu C, Hardee D, & Minnear F (2007) Membrane fusion by VAMP3 and plasma membrane t-SNAREs. Exp Cell Res 313(15):3198-3209; Veale K J, Offenhauser C, Whittaker S P, Estrella R P, & Murray R Z (2010) Recycling endosome membrane incorporation into the leading edge regulates lamellipodia formation and macrophage migration. Traffic 11(10):1370-1379. Hence, the inventors considered that VAMP3 on TfR vesicles may also interact with syntaxin 4 in HBMECs.

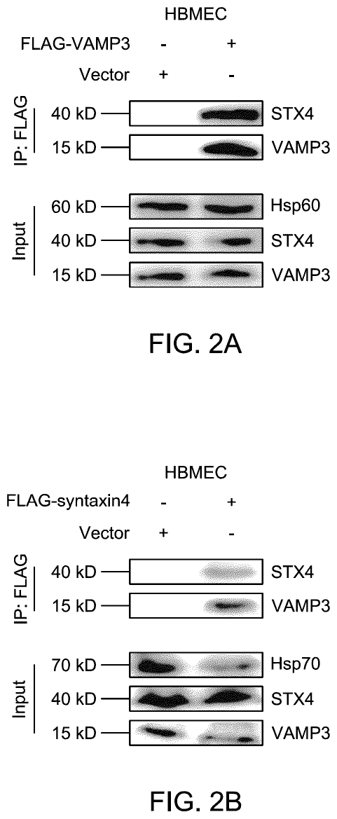

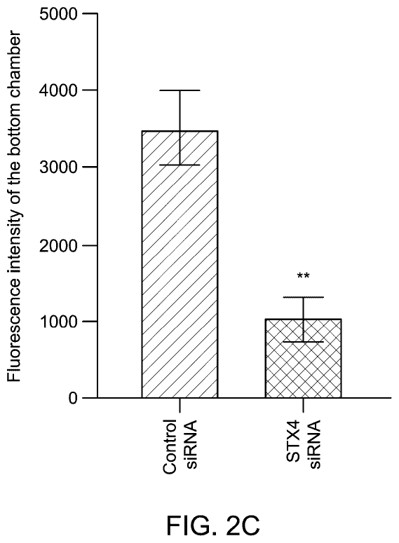

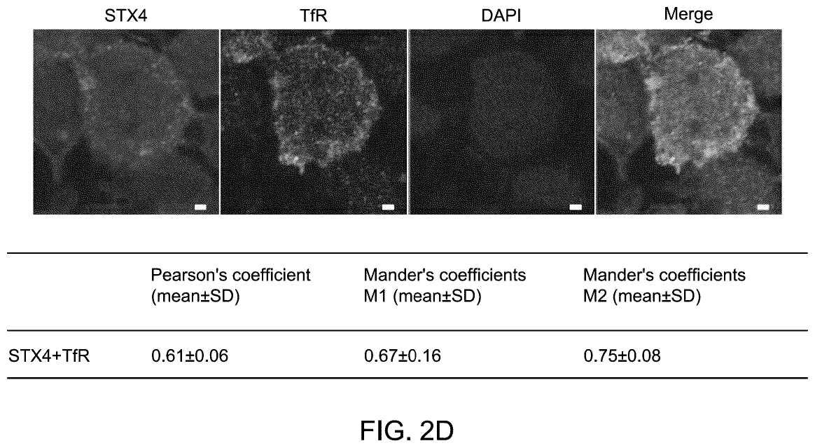

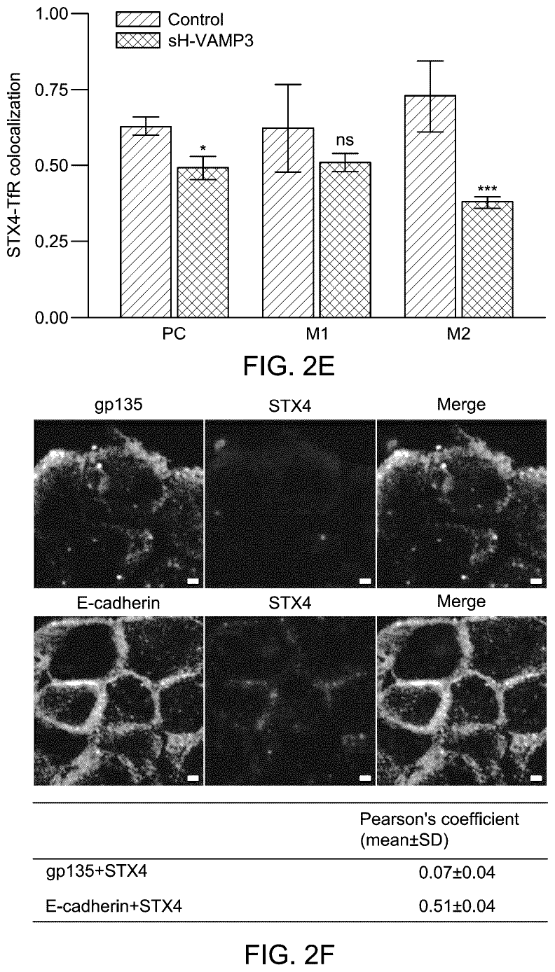

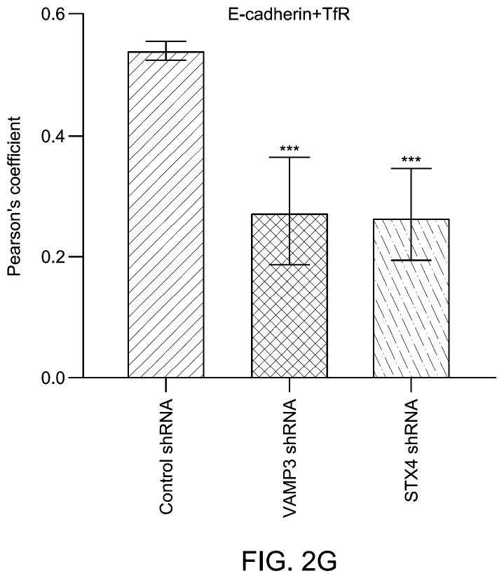

To evaluate this the inventors carried out co-immunoprecipitation assays using HBMECs transfected with FLAG-tagged VAMP3 or FLAG-tagged syntaxin 4. The results showed that VAMP3 was efficiently coimmunoprecipitated with syntaxin 4 (FIGS. 2A and B), indicating there is interaction between VAMP3 and syntaxin 4 within HBMECs.

Next, the inventors investigated whether syntaxin 4 also contributes to the transcytosis of Tf across HBMECs. They showed that silencing syntaxin 4 (reduction by 97%, FIG. 9) significantly reduced the amount of FITC-Tf transcytosed from the apical side (facing the lumen) to the basolateral side of polarized HBMEC monolayer in Transwells (FIG. 2C). Using confocal microscopy, colocalization of syntaxin 4 with TfR was observed in polarized HBMECs. FIG. 2D, Pearson's coefficient was 0.61, Manders' coefficient M1 was 0.67, Manders' coefficient M2 was 0.75.

Their colocalization was decreased in HBMECs with stable VAMP3-knock down using lentivirus compared with control cells (FIG. 2E), indicating the colocalization of syntaxin 4 with TfR is dependent on VAMP3.

Collectively, these data indicate that syntaxin 4 interactions with VAMP3 also plays a role in the transcytosis of Tf across HBMECs.

The residency of syntaxin 4 is reported be limited to the basolateral membrane in MDCK (Madin-Darby canine kidney) cell, a polarized epithelial cell line. Low S H, et al. (1996) Differential localization of syntaxin isoforms in polarized Madin-Darby canine kidney cells. Mol Biol Cell 7(12):2007-2018. The inventors next confirmed that syntaxin 4 is also located at the basolateral membrane of polarized HBMECs. Confocal microscopy analysis was used to analyze the location of syntaxin 4 in polarized HBMEC monolayer in Transwells, with the basolateral membrane marker E-cadherin and apical plasma membrane marker gp135. Only the colocalization of syntaxin 4 with E-cadherin was observed in polarized HBMECs (Pearson's coefficient was 0.51), and no intracellular staining of syntaxin 4 was detected (FIG. 2F). It indicates that syntaxin 4 that interacts with VAMP3 is located at the basolateral membrane of HBMECs.

Furthermore, using confocal microscopy, the inventors showed that these was a significant decrease in the colocalization of TfR with the basolateral membrane of polarized HBMECs with stable VAMP3 or syntaxin 4-knock down using lentivirus compared with control cells (FIG. 2G), indicating a decrease in the fusion of TfR vesicles with the basolateral membrane when VAMP3 or syntaxin 4 was silenced. Collectively, these data indicate that the interaction between VAMP3 on TfR vesicles and syntaxin 4 at the basolateral membrane of HBMECs, mediating the fusion of TfR vesicles with the basolateral membrane, is essential for the transcytosis of Tf across HBMECs.

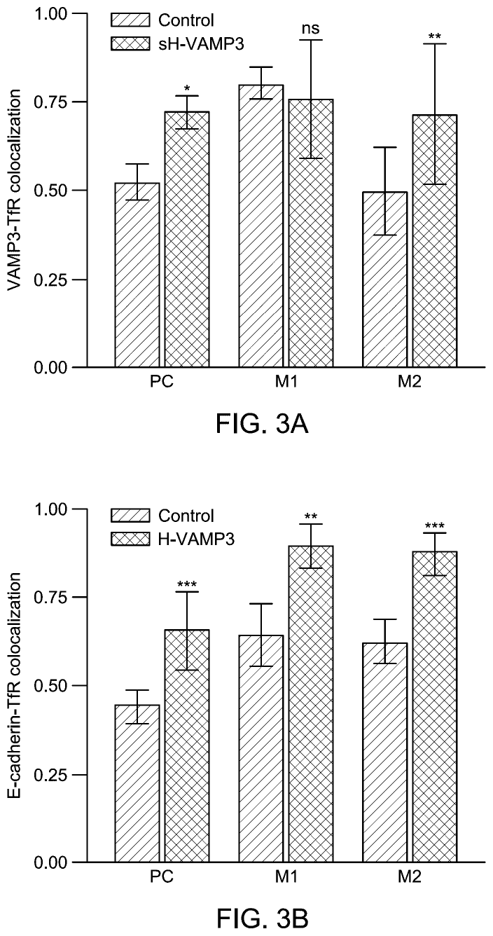

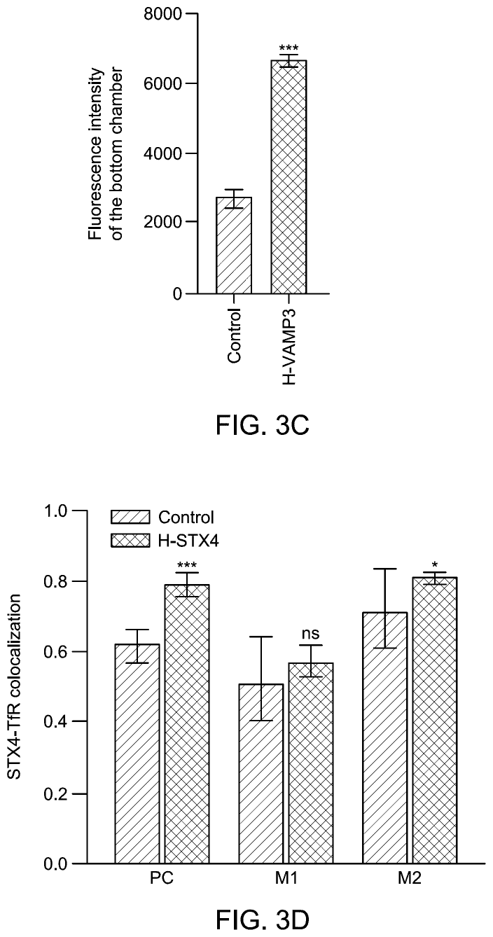

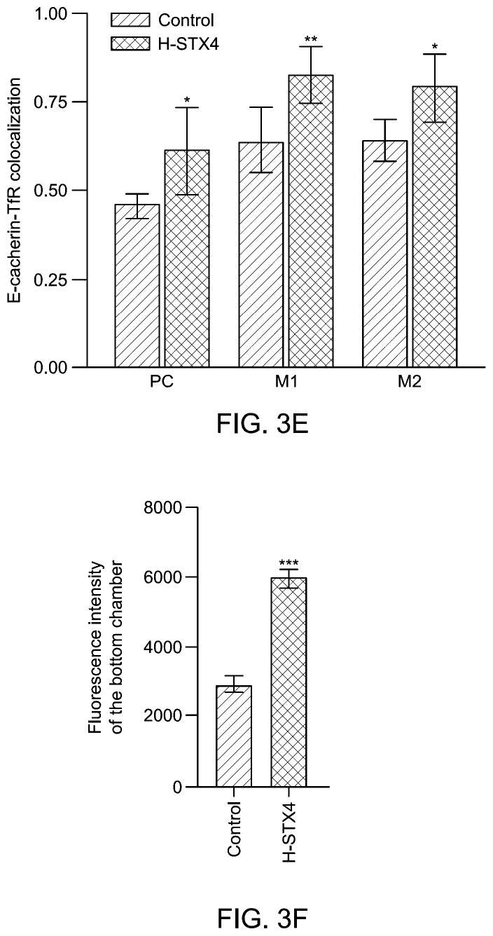

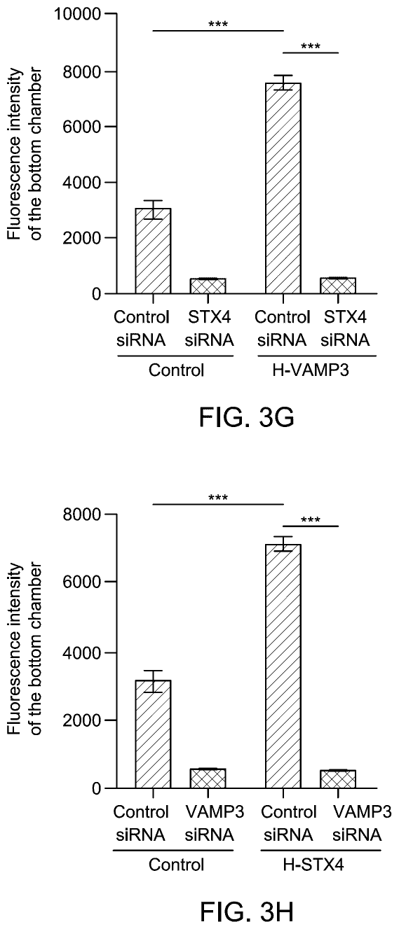

Overexpression of VAMP3 and syntaxin 4 enhances the efficiency of TfR transcytosis. As the above results showed that a small fraction of TfR vesicles within HBMECs are positive for VAMP3 (Manders' coefficient M2 was 0.45) (FIG. 1i), the inventors considered that increasing the proportion of TfR vesicles positive for VAMP3 by overexpressing VAMP3, can promote the fusion of TfR vesicles with the basolateral membrane, thereby enhancing the transcytosis of Tf across HBMECs. To test this hypothesis, the inventors stably overexpressed VAMP3 in HBMECs. Confocal microcopy showed that in HBMECs stably overexpressing VAMP3, the proportion of TfR positive for VAMP3 increased compared with control cells (M2 increased from 0.50 to 0.72) (FIG. 3A).

Furthermore, the colocalization of TfR with the basolateral membrane was also increased in HBMECs overexpressing VAMP3 (M2, the fraction of TfR colocalized with E-cadherin, increased from 0.62 to 0.87) (FIG. 3B).

Additionally, the inventors showed that the efficiency of Tf transcytosis through HBMECs stably overexpressing VAMP3 was enhanced by 2.46-fold compared with control cells (FIG. 3C). These data indicate that increasing VAMP3 expression enhances the proportion of TfR vesicles carrying VAMP3, leading to increased fusion between TfR vesicles and the basolateral membrane and, consequently, a higher efficiency of TfR transcytosis.

As VAMP3 interacts with syntaxin 4 to mediate the fusion of TfR vesicles with the basolateral membrane, the inventors considered that overexpressing syntaxin 4 also can promote the transcytosis of Tf across HBMECs. The proportion of TfR overlapping syntaxin 4 was increased compared with control cells where M2 increased from 0.72 to 0.81 as shown in FIG. 3D.

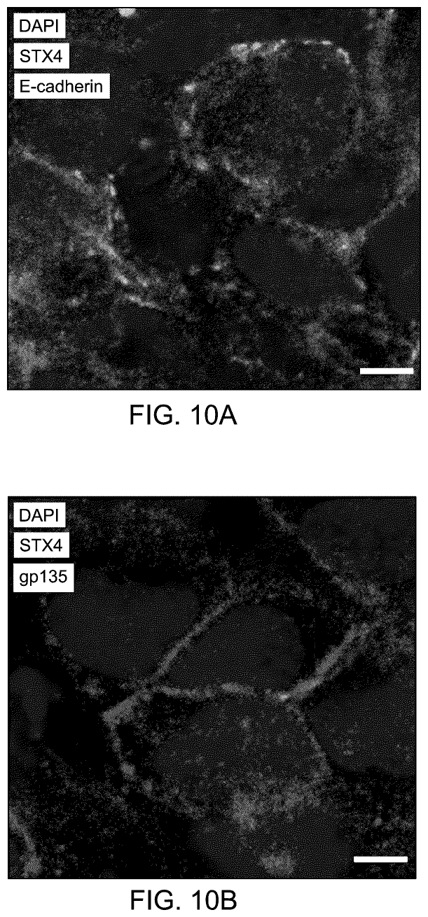

As shown by the results in FIGS. 10A and 10B, STX-4 was only present at the basolateral membrane of H-STX-4. Confocal microcopy showed that in HBMECs stably overexpressing syntaxin 4 which was also only present at the basolateral membrane.

In addition, the colocalization of TfR with the basolateral membrane was also increased in HBMECs overexpressing syntaxin 4 (M2 increased from 0.65 to 0.79) (FIG. 3E). Consistently, the transcytosis of Tf through HBMECs stably overexpressing syntaxin 4 was enhanced by 2.02-fold comparted with control cells (FIG. 3F). However, the inventors showed that silencing VAMP3 or syntaxin 4 inhibited the beneficial effect of overexpressing syntaxin 4 or VAMP3, respectively, on Tf transcytosis (FIGS. 3G and H).

It is inconsistent with that VAMP3 and syntaxin 4 function collaboratively to mediate the TfR transcytosis across HBMECs. Collectively, these data indicate that overexpression of VAMP3 and syntaxin 4 promotes the fusion of TfR vesicles with the basolateral membrane, and thus increasing the efficiency of TfR transcytosis across HBMECs.

### VAMP3 and Syntaxin 4 Contribute to the Transcytosis of NMEC

Next, as our recent work showed that NMEC couple its penetration of BBB to TfR transcytosis, the inventors investigated whether VAMP3 on TfR vesicles and syntaxin 4 at the basolateral membrane of HBMECs also contributes to the transcytosis of NMEC. Cheng Z, et al. (2023) Pathogenic bacteria exploit transferrin receptor transcytosis to penetrate the blood-brain barrier. Proc Natl Acad Sci USA 120(39):e2307899120.

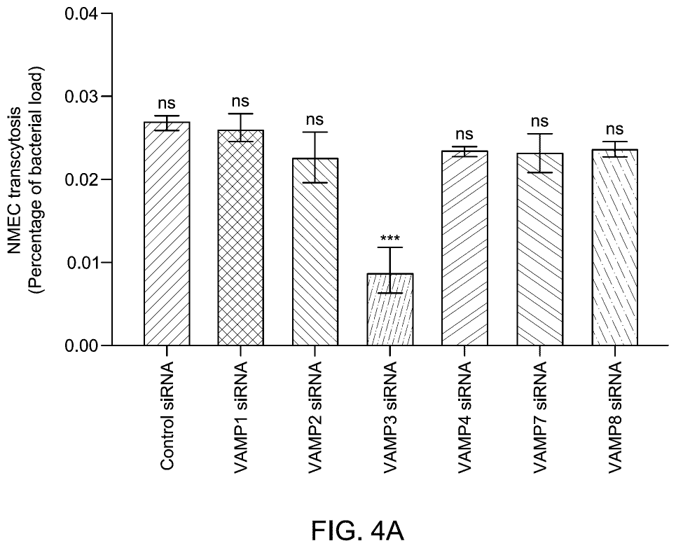

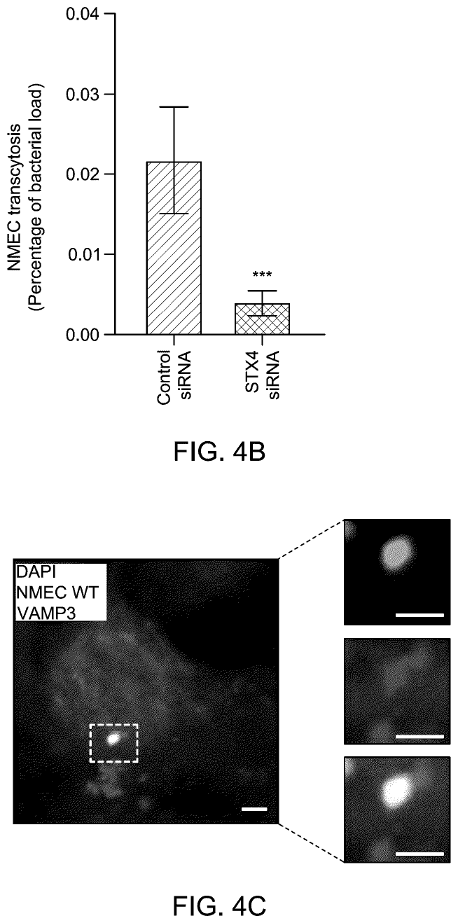

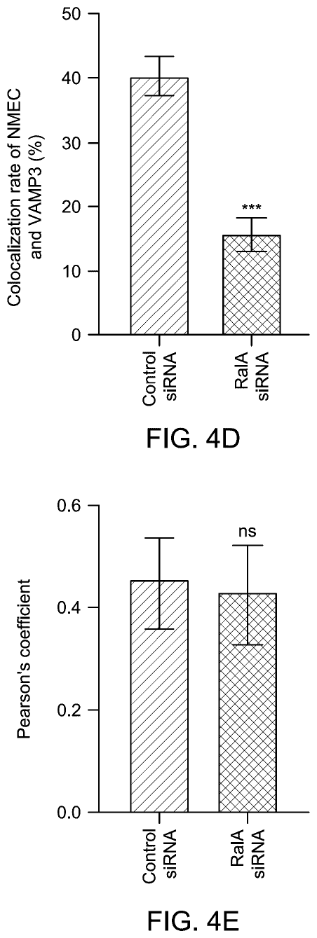

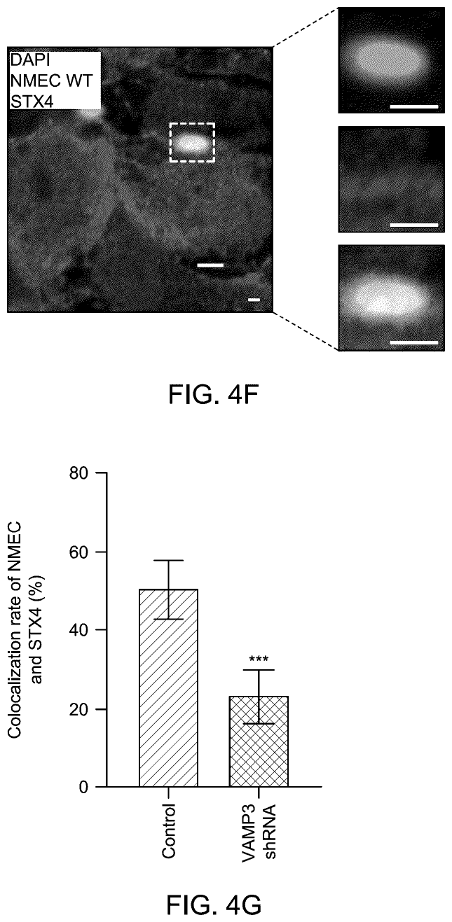

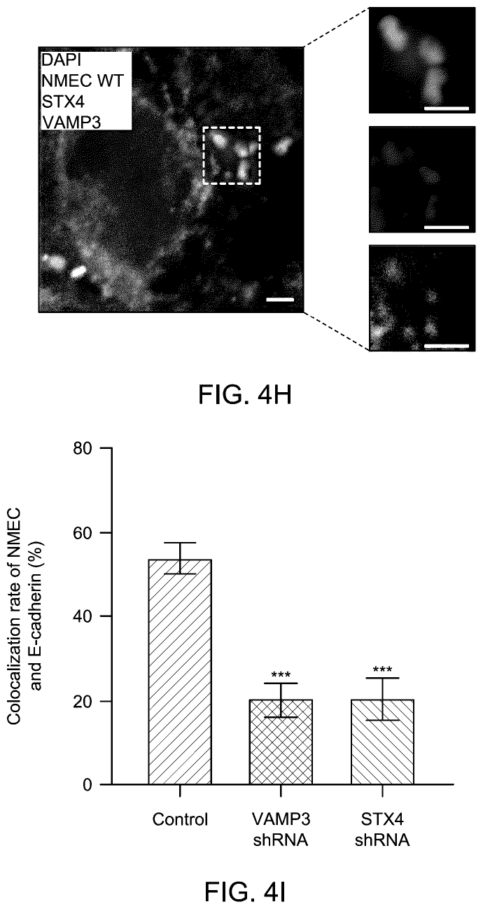

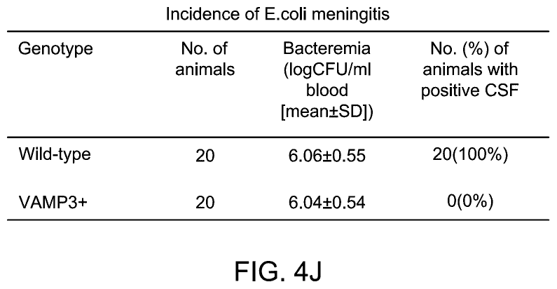

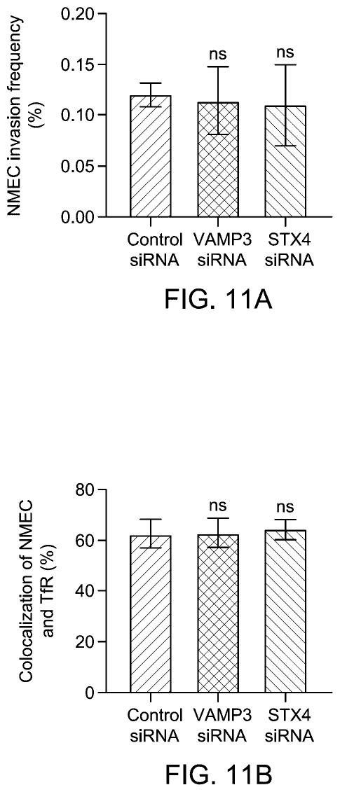

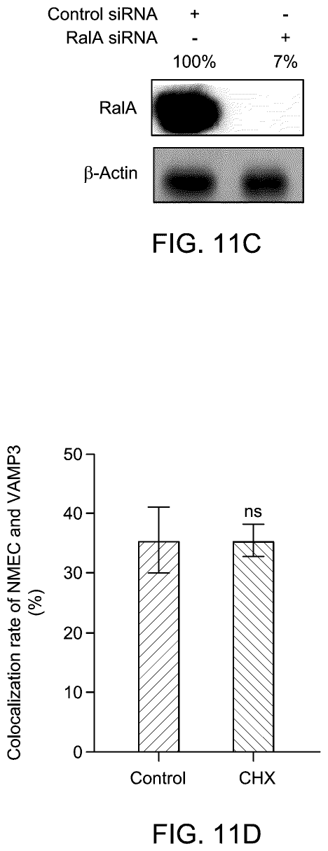

The inventors showed that silencing VAMP3 or syntaxin 4 significantly reduced the transcytosis of NMEC across the polarized HBMEC monolayer in Transwells in vitro (FIGS. 4A and B). In contrast, silencing VAMP3 or syntaxin 4 had no effect on the bacterial invasion of HBMECs (FIG. 11A) and the fusion of BCVs with TfR vesicles within HBMECs (FIG. 11B). It indicates that VAMP3 and syntaxin 4 contribute to the transcytosis of NMEC across HBMECs.

Confocal microcopy showed that 40.3% BCVs were colocalized with VAMP3 (FIG. 4C). As the above results showed that VAMP3 is present on TfR vesicles in HBMECs, the inventors investigated whether the colocalization of VAMP3 with BCVs depends on the fusion of BCVs and TfR vesicles.

The inventors showed that silencing RalA (reduction by 93%, FIG. 11C), which is known to decrease the fusion of BCVs and TfR vesicles, significantly reduced the colocalization of VAMP3 with BCVs (FIG. 4D). Cheng Z, et al. (2023) Pathogenic bacteria exploit transferrin receptor transcytosis to penetrate the blood-brain barrier. Proc Natl Acad Sci USA 120(39):e2307899120.

In contrast, silencing RalA did not influence the colocalization of VAMP3 with TfR (FIG. 4E). In addition, the inventors showed that inhibition of protein synthesis in HBMECs prior to NMEC infection had no effect on the colocalization of VAMP3 with BCVs (FIG. 11D). These data indicate that VAMP3 colocalized with BCVs originates from TfR vesicles in HBMECs.

Furthermore, confocal microcopy showed that 51.2% of BCVs were colocalized with syntaxin 4 in polarized HBMECs (FIG. 4F). The inventors found that there was a significant decrease in colocalization between BCVs and syntaxin 4 in polarized HBMECs with stable VAMP3-knock down compared with control cells (FIG. 4G), indicating that the interaction between BCVs and syntaxin 4 depends on VAMP3. Consistently, the colocalization of VAMP3, syntaxin 4 and BCVs was observed at the basolateral membrane of polarized HBMECs (FIG. 4H).

Furthermore, the inventors observed that the percent of NMEC colocalized with the basolateral region of infected HBMECs with stable VAMP3-knock down or syntaxin 4-knock down exhibited a significant decrease compared with control cells (FIG. 4I). It indicates that VAMP3 and syntaxin 4 mediates the fusion of TfR-NMEC vesicles with the basolateral membrane of HBMECs.

As the disruption of syntaxin 4 results in early embryonic lethality of mice, the inventors used gene-deficient VAMP3^{-/-} mice to perform the animal experiments. Yang C, et al. (2001) Syntaxin 4 heterozygous knockout mice develop muscle insulin resistance. J Cin Invest 107(10):1311-1318. The results showed that VAMP3^{-/-} mice exhibited significantly reduced bacterial titers in the cerebrospinal fluid after tail vein infection compared to wild-type mice (FIG. 4J), indicating the transcytosis of NMEC across the BBB was inhibited in VAMP3^{-/-} mice.

Collectively, these data indicate that VAMP3 and syntaxin 4 contributes to the transcytosis of NMEC across the BBB by mediating the fusion of TfR-NMEC vesicles with the basolateral membrane of HBMECs.

### NMEC Increases its Transcytosis Efficiency by Enhancing the Expression of VAMP3 and Syntaxin 4

The expression of VAMP3 and syntaxin 4 is both induced in response to LPS in macrophage. Pagan J K, et al. (2003) The t-SNARE syntaxin 4 is regulated during macrophage activation to function in membrane traffic and cytokine secretion. Curr Biol 13(2):156-160; Murray R Z, Kay J G, Sangermani D G, & Stow J L (2005) A role for the phagosome in cytokine secretion. Science 310(5753):1492-1495. The inventors considered that infection of HBMECs by NMEC would induce the expression of VAMP3 and syntaxin 4 via LPS.

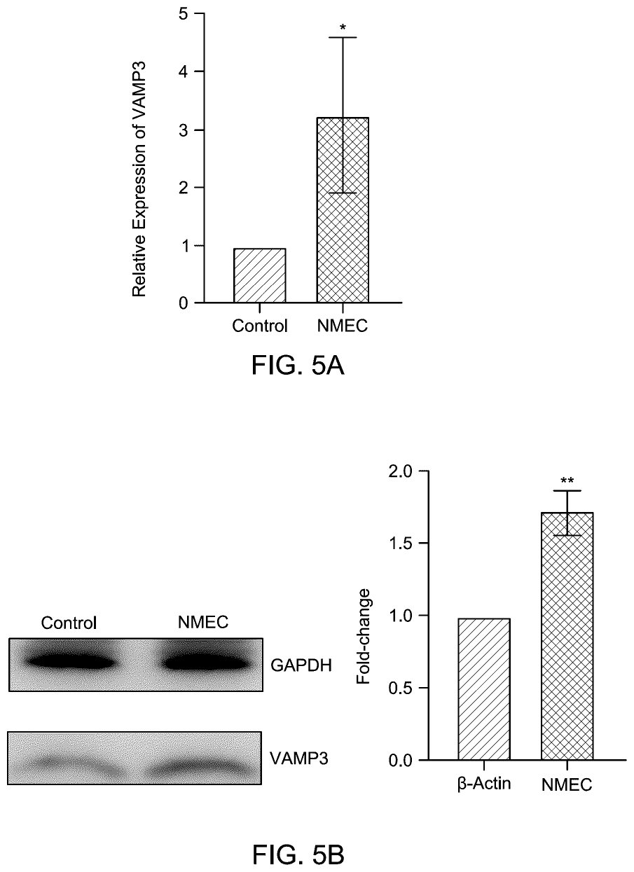

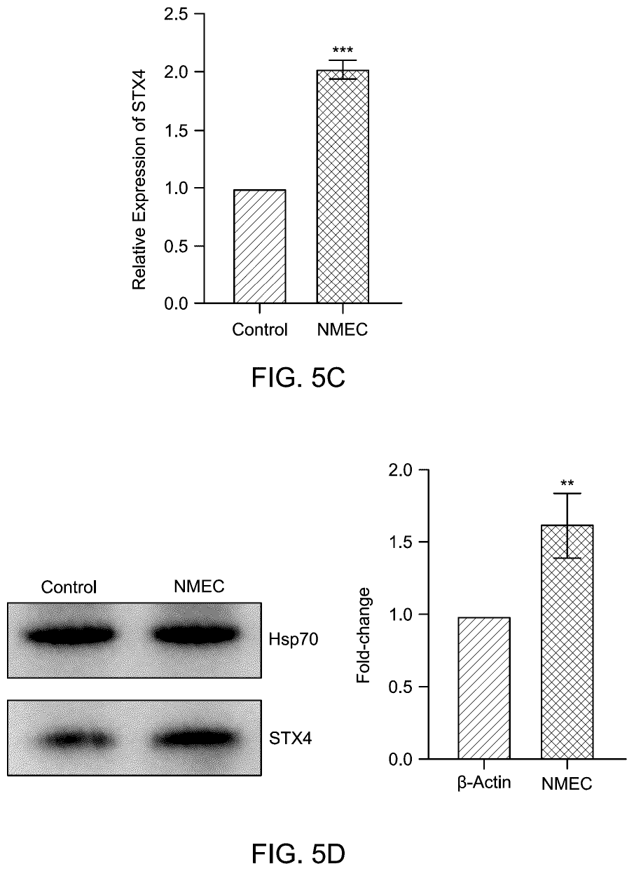

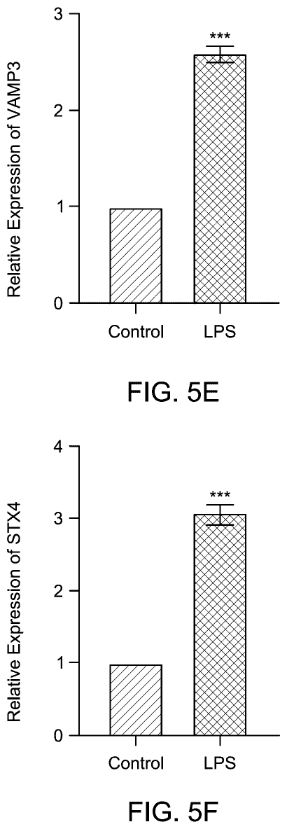

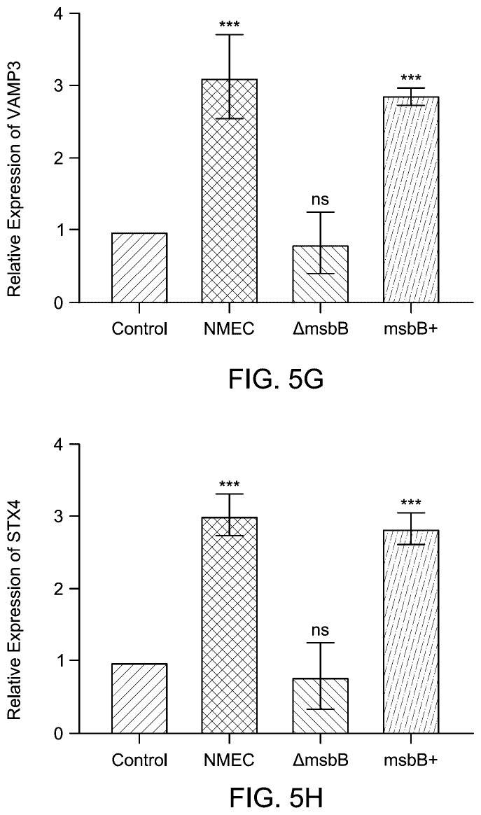

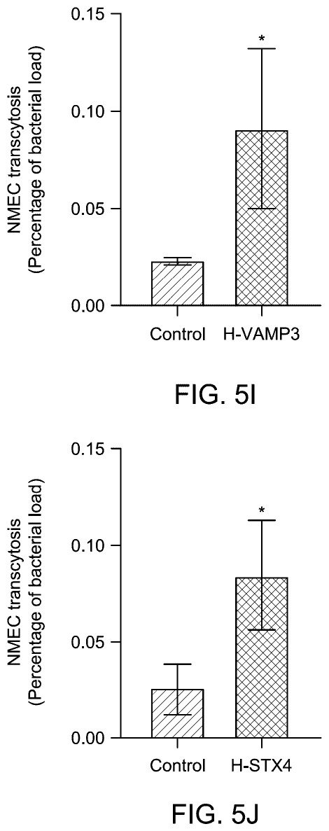

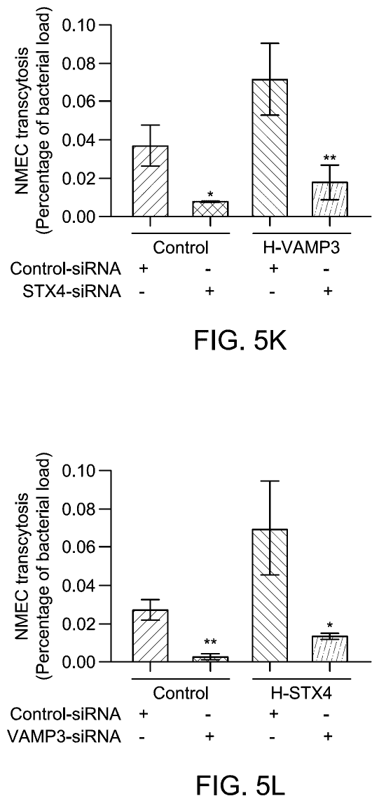

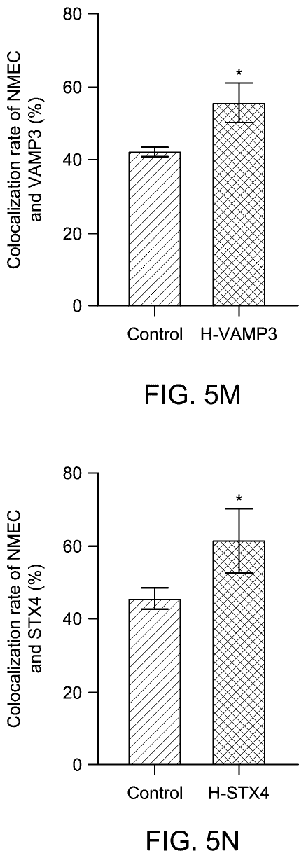

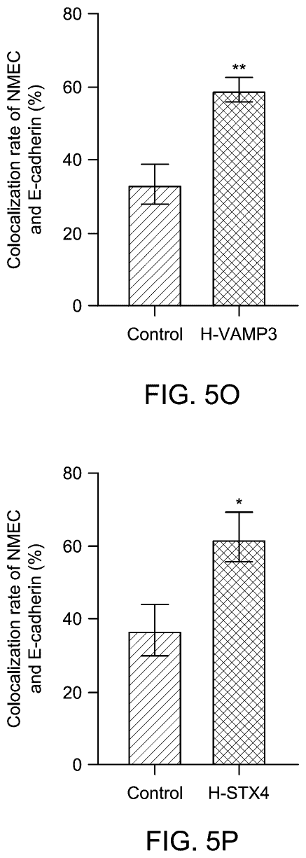

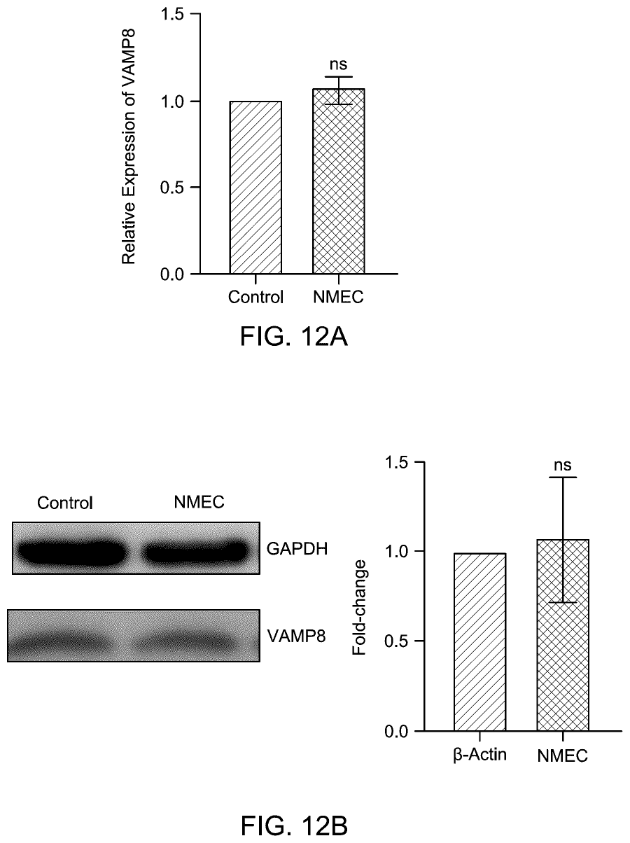

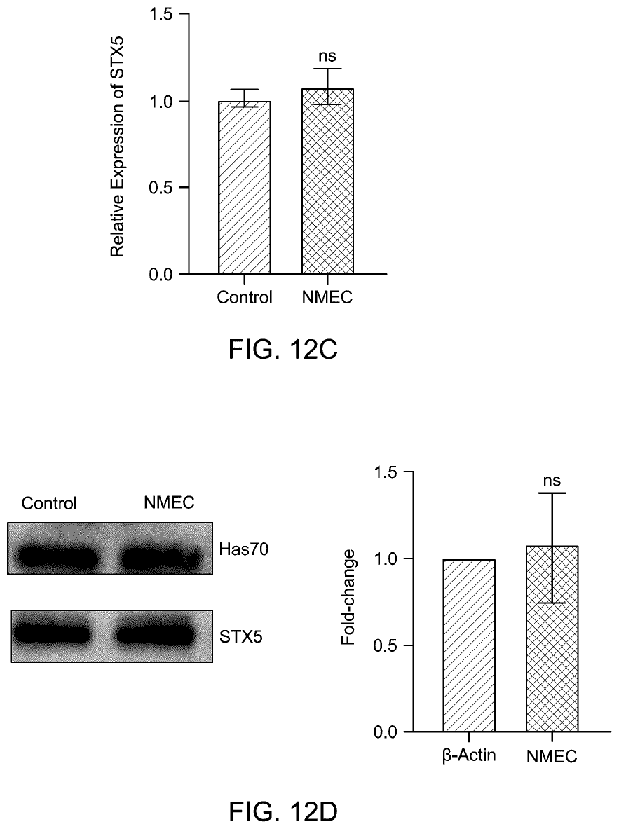

To explore this idea, the inventors analyzed the expression of VAMP3 and syntaxin 4 in response to NMEC infection. qRT-PCR and western blotting assays showed that the expression of VAMP3 and syntaxin 4 is upregulated in NMEC-infected HBMECs (FIGS. 5A-D). However, as controls, the expression of VAMP8 and syntaxin 5, which are also present on the BCVs in HBMECs, exhibited no significant change in response to NMEC infection (FIGS. 12A-12D). Cheng Z, et al. (2023) Pathogenic bacteria exploit transferrin receptor transcytosis to penetrate the blood-brain barrier. Proc Natl Acad Sci USA 120(39):e2307899120.

Additionally, the inventors showed that treatment of HBMECs by LPS results in the up regulation of VAMP3 and syntaxin 4 (FIGS. 5E and 5F).

In contrast, infection of HBMECs with the NMEC mutant ΔmsbB, which lacks LPS, did not induce the expression of VAMP3 and syntaxin 4 (FIGS. 5G and 5H). Complementation of ΔmsbB by wild type msbB restored the ability of bacteria to induce VAMP3 and syntaxin 4 expression (FIGS. 5G and 5H). These data indicate that NMEC infection induce the expression of VAMP3 and syntaxin 4 via LPS.

Next, the inventors investigated the effect of increased expression of VAMP3 and syntaxin 4 on NMEC transcytosis. They showed that the transcytosis of NMEC through HBMECs stably overexpressing VAMP3 or syntaxin 4 was significantly enhanced comparted with control cells (FIGS. 5I and 5J). However, silencing VAMP3 or syntaxin 4 inhibited the influence of overexpressing syntaxin 4 or VAMP3 on NMEC transcytosis (FIGS. 5K and 5L). It is in consistent with that VAMP3 and syntaxin 4 function collaboratively to mediate the transcytosis of NMEC. These data indicate that enhanced expression of VAMP3 and syntaxin 4 in response to NMEC infection promotes bacterial transcytosis.

Furthermore, investigated how the overexpression of VAMP3 and syntaxin 4 increase the transcytosis of NMEC. Confocal microcopy showed that the percent of BCVs colocalized with VAMP3 or syntaxin 4 were increased in HBMECs that stably overexpresses VAMP3 or syntaxin 4 compared with control cells (FIGS. 5M and 5N). The inventors also found that the colocalization of BCVs with the basolateral membrane was increased in HBMECs overexpressing VAMP3 or syntaxin 4 (FIGS. 5O and 5P). These data indicate that the overexpression of VAMP3 or syntaxin 4 increase the proportion of BCVs positive for VAMP3 or syntaxin 4, and this benefits the fusion of BCVs with the basolateral membrane of HBMECs, leading to the enhanced transcytosis of NMEC.

### The Expression of VAMP3 and Syntaxin 4 Expression is Induced Via TLR4-TRIF-Dependent Signaling Pathway.

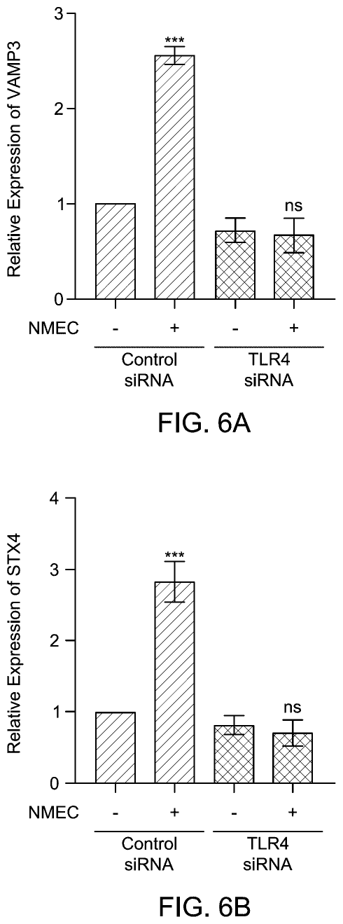

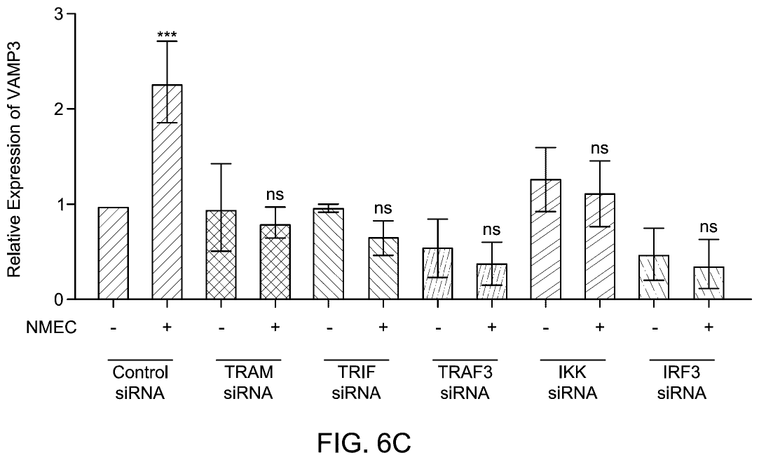

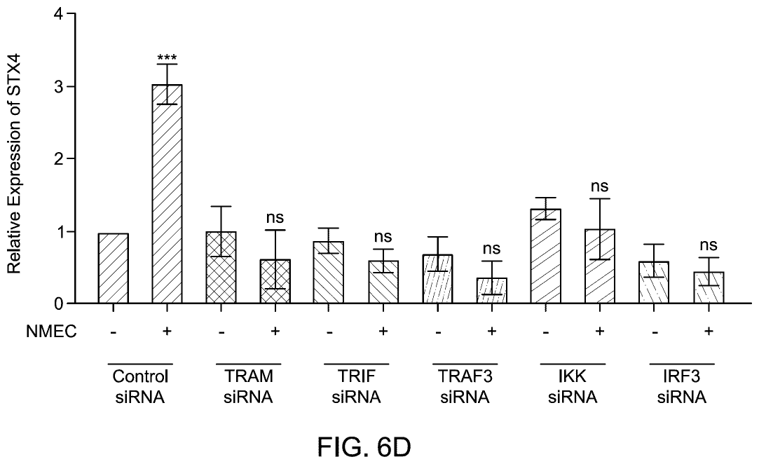

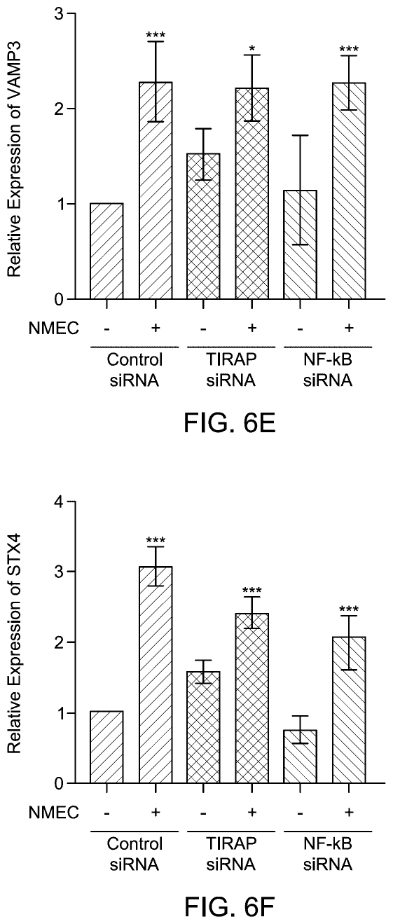

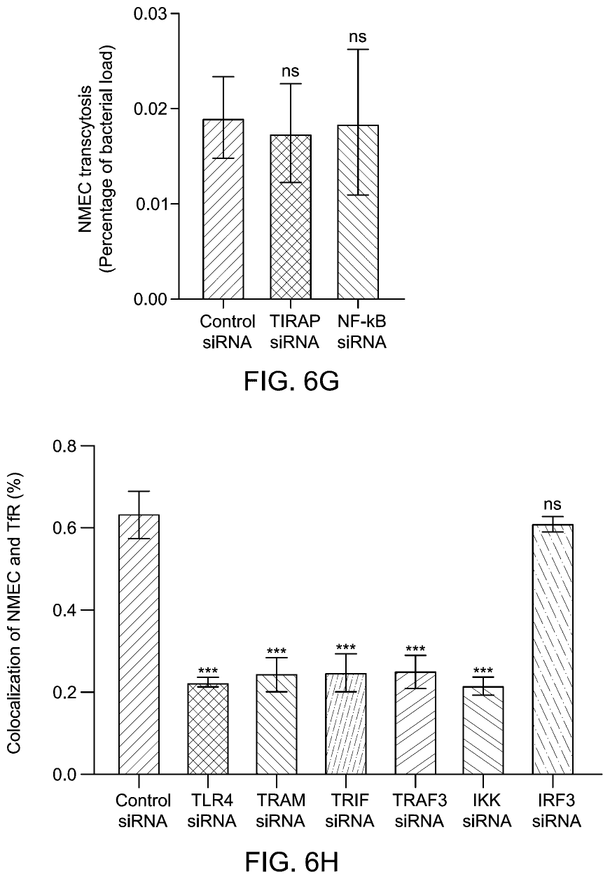

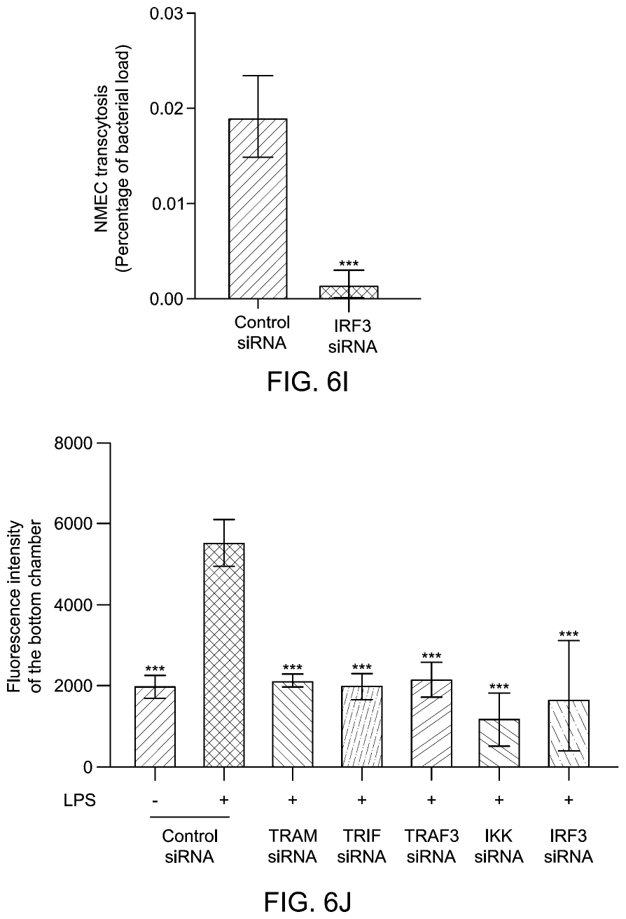

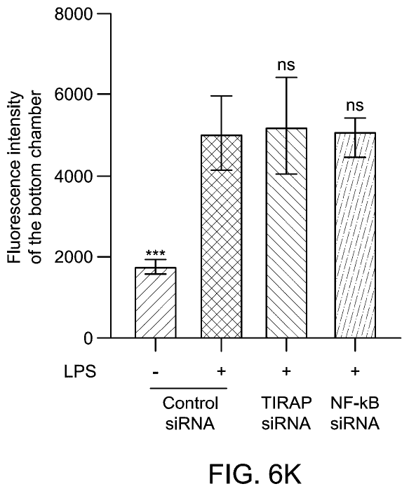

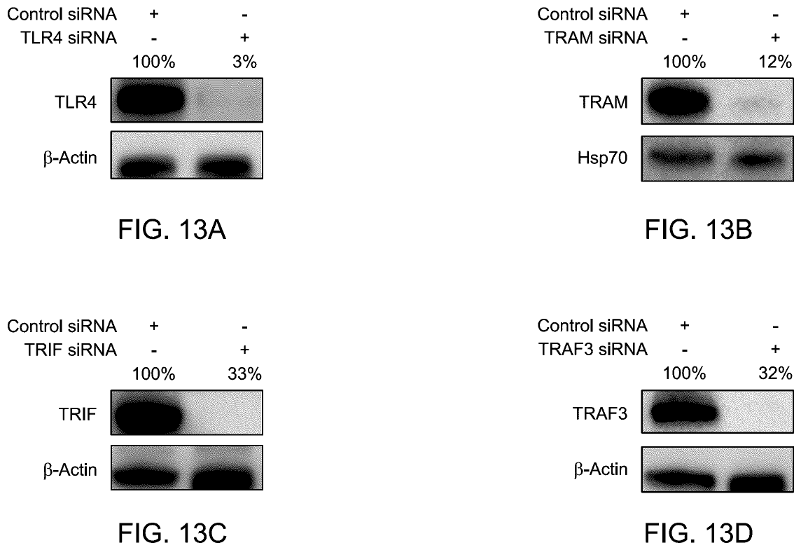

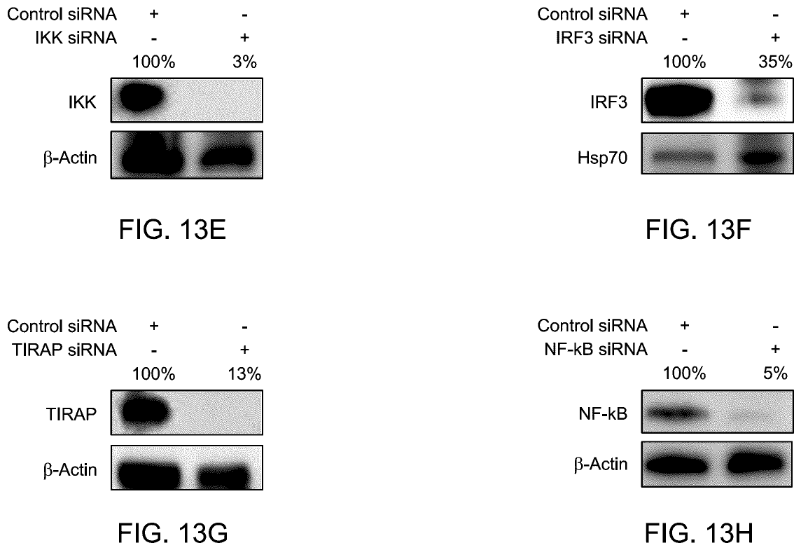

Although it is known that LPS induces the expression of VAMP3 and syntaxin 4 in macrophage, the underlying mechanism remains unclear. Pagan J K, et al. (2003) The t-SNARE syntaxin 4 is regulated during macrophage activation to function in membrane traffic and cytokine secretion. Curr Biol 13(2):156-160; Murray R Z, Kay J G, Sangermani D G, & Stow J L (2005) A role for the phagosome in cytokine secretion. Science 310(5753):1492-1495. LPS of Gram-negative bacteria can activate TLR4 signaling of host cell. In consistent, the inventors showed that the upregulation of VAMP3 and syntaxin 4 expression in response to NMEC infection was inhibited when TLR4 in HBMECs was silenced (reduction by 97%, FIG. 13) (FIGS. 6A and 6B).

TLR4 activates two distinct signaling pathways: the TIRAP-dependent pathway (TLR4-TIRAP-NFκB) and the TRIF-dependent pathway (TLR4-TRAM-TRIF-TRAF3-IKK-IRF3). Ciesielska A, Matyjek M, & Kwiatkowska K (2021) TLR4 and CD14 trafficking and its influence on LPS-induced pro-inflammatory signaling. Cell Mol Life Sci 78(4):1233-1261.

Next, the inventors investigated which pathway is activated by LPS and TLR4 to induce the expression of VAMP3 and syntaxin 4 in HBMECs. They showed that the upregulation of VAMP3 and syntaxin 4 expression in response to NMEC infection was inhibited when TRAM, TRIF, TRAF3, TKK or IRF3 in HBMECs was silenced (reduction by 65-97%, FIGS. 6C and 6D), but not influenced when TIRAP or NFκB was silenced (reduction by 87-95%, FIG. 13) (FIGS. 6E and 6F). It indicates that VAMP3 and syntaxin 4 expression in HBMECs is induced by LPS via TLR4-TRIF-dependent pathway, but not the TLR4-TIRAP-dependent pathway.

Next, the inventors showed that the transcytosis of NMEC across HBMECs was not influenced when factors involved in the TIRAP-dependent pathway (TIRAP and NFκB) were silenced (FIG. 6G). It is in consistent with that TIRAP-dependent pathway does not contribute to the induction of VAMP3 and syntaxin 4 expression in response to NMEC infection.

Our previous result showed that several factors within TRIF-dependent pathway, including TLR4, TRAM, TRIF and TRAF3, are also mediate the fusion of BCV and TfR vesicle in HBMECs. Cheng Z, et al. (2023) Pathogenic bacteria exploit transferrin receptor transcytosis to penetrate the blood-brain barrier. Proc Nat Acad Sci USA 120(39):e2307899120. It indicates that they may contributes to the transcytosis of NMEC by influencing the fusion of BCV and TfR vesicles. Different from these factors, the inventors showed that silence of IRF3, which is also involved in TRIF-dependent pathway, did not influence the fusion of BCV and TfR vesicles (FIG. 6H). Then, the inventors investigated whether IRF3 influences the transcytosis of NMEC across HBMECs. The results showed that when IRF3 was silenced, the transcytosis of bacteria was significantly inhibited (FIG. 6I). It indicates that TRIF-dependent pathway promotes the transcytosis of NMEC by regulating the expression of VAMP3 and syntaxin 4.

Furthermore, the inventors showed that treatment of HBMECs by LPS, which leads to the increased expression of VAMP3 and syntaxin 4, promoted the transcytosis of Tf (FIGS. 5E and 5F), and this effect was inhibited when TLR4-TRIF-dependent pathway was blocked, but not influenced when TIRAP-dependent pathway was blocked (FIGS. 6J and 6K). It is in consistent with that VAMP3 and syntaxin 4 expression is induced in response to LPS via TLR4-TRIF-dependent pathway, but not the TLR4-TIRAP-dependent pathway

### IRF3 in the TLR4-TRIF-Dependent Pathway Directly and Indirectly Regulates the Expression of VAMP3 and Syntaxin 4 Respectively.

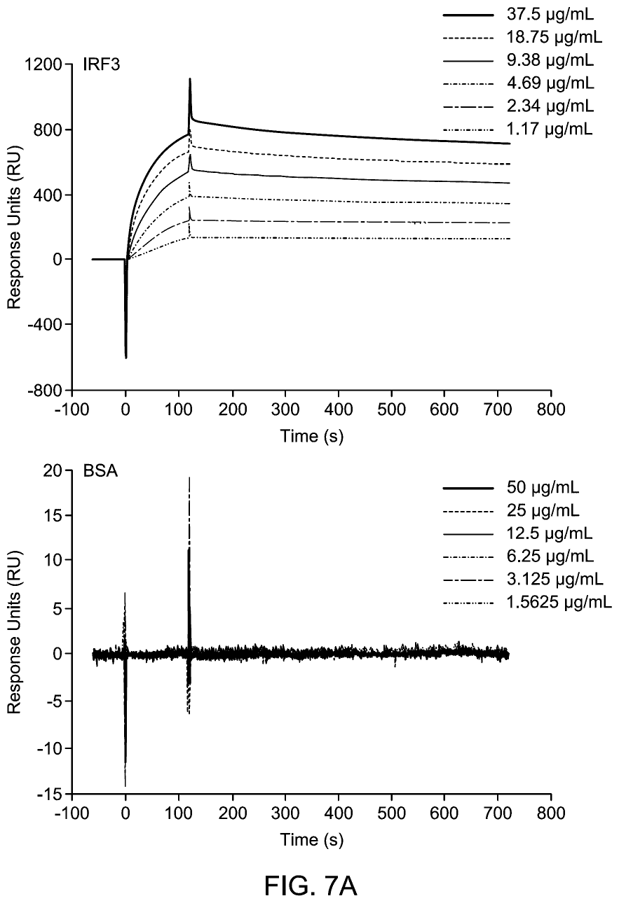

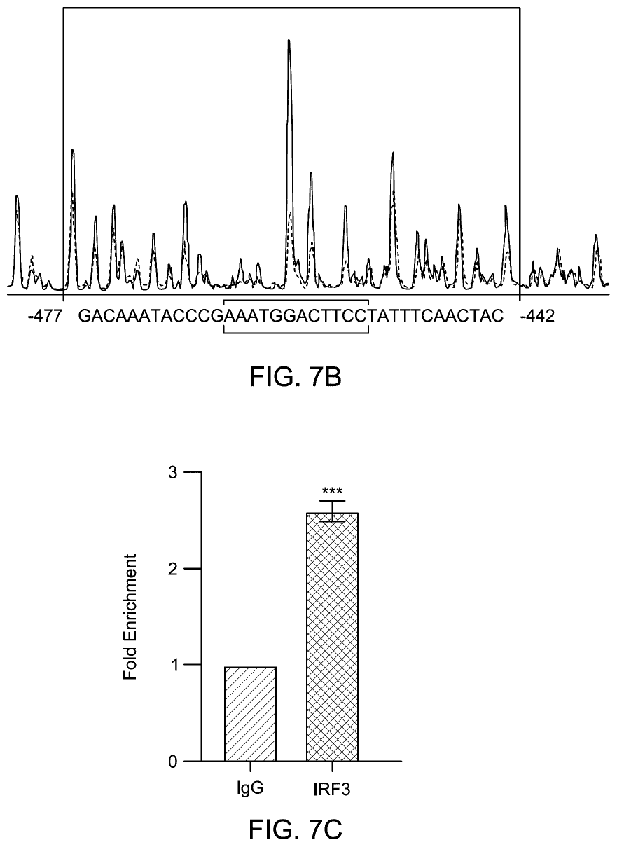

IRF3 is a regulator absolutely required for the induction of IFN-β and certain IFN-α species. Liu Y P, et al. (2012) Endoplasmic reticulum stress regulates the innate immunity critical transcription factor IRF3. J. Immunol. 189(9):4630-4639; Sakaguchi S, et al. (2003) Essential role of IRF-3 in lipopolysaccharide-induced interferon-beta gene expression and endotoxin shock. Biochem Biophys Res Commun 306(4):860-866. It also regulates other inflammatory mediators such as the chemokines CXCL10 and RANTES. Lu X, Masic A, Liu Q, & Zhou Y (2011) Regulation of influenza A virus induced CXCL-10 gene expression requires PI3K/Akt pathway and IRF3 transcription factor. Mol. Immunol. 48(12-13):1417-1423; McWhirter S M, et al. (2004) IFN-regulatory factor 3-dependent gene expression is defective in Tbkl-deficient mouse embryonic fibroblasts. Proc Natl Acad Sci USA 101(1):233-238. Therefore, the inventors tried to investigate whether IRF3 directly regulates the expression of VAMP3 and syntaxin 4. Surface plasmon resonance assays using Biacore X100 SPR showed that 6×His-tagged IRF3 interacts with the promoter region of VAMP3, but not the syntaxin 4 promoter (FIG. 7A). Using a dye-based DNase I foot-printing assay, the inventors revealed a specific IRF3-bound sequence containing a 12-base pair motif (5-AAATGGACTTCC-3)(SEQ ID NO: 5) in the promoter region of VAMP3 (FIG. 7B), which is located −465 bp to −454 bp from the proximal transcriptional start site and exhibits similarity to the reported motif bound by IRF3. Morin P, et al. (2002) Preferential binding sites for interferon regulatory factors 3 and 7 involved in interferon-A gene transcription. J Mol. Biol. 316(5):1009-1022.

The inventors also performed ChIP-qPCR assays to analyze the enrichment of the VAMP3 promoter in IRF3-ChIP samples compared with mock-ChIP control. The results showed that the VAMP3 promoter was significantly enriched in DNA binding to IRF3 compared to control DNA (FIG. 7C). These data indicate that IRF3 directly regulates the expression of VAMP3 by binding to its promoter. However, the regulation of syntaxin 4 expression by IRF3 is indirect, and its underlying mechanism could be a subject of future research.

Terminology. Terminology used herein is for the purpose of describing particular embodiments only and is not intended to be limiting of the invention.

The term “we” refers to the inventors.

Unless expressly stated, the terms used herein are intended to have the plain and ordinary meaning as understood by those of ordinary skill in the art.

The following definitions are intended to aid the reader in understanding the present disclosure but are not intended to vary or otherwise limit the meaning of such terms unless specifically indicated.

While aspects of the present disclosure have been described in conjunction with the specific embodiments thereof that are proposed as examples, alternatives, modifications, and variations to the examples may be made. The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

It should be noted that, as used in the specification and the appended claims, the singular forms “a,” “an,” and “the” include plural referents unless the context clearly dictates otherwise. See Harari v. Lee, 656 F.3d 1331, 1341, (Fed. Cir. 2011); Baldwin Graphic Sys., Inc. v. Siebert, Inc., 512 F.3d 1338, 1342 (Fed. Cir. 2008)); KJC Corp. v. Kinetic Concepts, Inc., 223 F.3d 1351, 1356 (Fed. Cir. 2000).

As used herein, the term “and/or” includes any and all combinations of one or more of the associated listed items and may be abbreviated as “/”. A and/or B includes A, B, and (A+B).

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−0.2% of the stated value (or range of values), +/−0.5% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges and values subsumed therein.

Any numerical range recited herein is intended to include all sub-ranges and values subsumed therein. Where a range of values is provided, it is to be understood that each intervening value between an upper and lower limit of the range and any other stated or intervening value in that stated range is encompassed within the disclosure. Where the stated range includes upper and lower limits, ranges excluding either of those limits are also included.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-2, 1-3, 1-4, 1-5, 1-6, 1-7, 1-8, 1-9, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5- 10, 6-7, 6-8, 6-9, 6-10, 7-8, 7-9, 7-10, 8-9, 8-10, 9-10 as some examples. A range encompasses its endpoints as well as values inside of an endpoint, for example, the range 0-5 includes 0, >0, 1, 2, 3, 4, <5 and 5.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Modulating refers to increasing or decreasing a level of a molecule, such as a level of VAMP3 or STX-4 in a cell or vesicle or increasing or decreasing a rate of transcription or translation of nucleic acids encoding VAMP3 or STX-4. This may involve altering the rate of transcription of a gene encoding VAMP3 or STX-4 or the translation of mRNA encoding VAMP3 or STX-4 into protein, for example by altering transcription factors, enhancers or other regulatory elements.

In some embodiments, modulation refers to increasing or over-expressing an amount of VAMP3 or STX-4 in a cell or vesicle by 1, 2, 5, 10, 20, 50, 100, 200 or >200% compared to an untreated control.

In other embodiments modulation refers to reducing the expression of VAMP3 or STX-4 from mRNA, for example by 1, 2, 5, 10, 20, 50, <100 or 100% compared to an untreated control.

This term also refers to increasing or decreasing the amount or rate of transcytosis of a target molecule, such as a pharmaceutical or biologic drug or agent. In some embodiments, the methods disclosed herein may increase transcytosis >1, 2, 2.5, 3, 3.5, 4, 4.5, 5, 5.5, 6, 6.5, 7 or >7 fold compared to a control cell not treated as disclosed herein. In other embodiments modulation refers to reducing transcytosis, for example by 1, 2, 5, 10, 20, 50, <100 or 100%. Transcytosis efficiency is advantageously measured using an in vitro transwell assay based on relative fluorescence. However, relative expression or amount of VAMP3 or STX-4 may also be measured as a wt. % or mole % as applied to the ranges described above.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

1. A method for modulating transcytosis of a target molecule in a cell comprising modulating Vesicle-associated membrane protein 3 (VAMP3) and/or Syntaxin 4 (STX-4) expression or modulating levels of VAMP3 or STX-4.

2. The method of claim 1, wherein the cell is a Human Brain Microvascular Endothelial Cell (HBMEC).

3. The method of claim 1, wherein VAMP3 expression or cellular levels of VAMP3 are increased in a HBMEC compared to otherwise similar HBMCs not treated to increase VAMP3 expression or VAMP3 level.

4. The method of claim 1, wherein VAMP3 expression or VAMP3 levels is increased by administering a drug, peptide or polynucleotide that increases VAMP3 expression.

5. The method of claim 3, wherein VAMP3 expression is increased by administering live, attenuated, or dead neonatal meningitis Escherichia coli (NMEC), Streptococcus pneumoniae, or group B Streptococcus, LPS, or components thereof which increases expression of VAMP3.

6. The method of claim 1, wherein level of VAMP 3 in HBMECs is increased by transiently or permanently transforming the HBMECs with a nucleic acid encoding VAMP3.

7. The method of claim 1, wherein VAMP3 level in HBMECs is increased by loading the HBMECs with exogenous VAMP3.

8. The method of claim 1, wherein VAMP3 level in HBMECs is increased by loading HBMEC vesicles comprising TfR with exogenous VAMP3.

9. The method of claim 1, wherein said transcytosis comprises movement of the target molecule or target molecule conjugate or complex that comprises an antibody or other protein molecule, or a conjugate thereof, from the blood compartment to a brain compartment.

10. The method of claim 1, wherein said transcytosis comprises movement of the target molecule or target molecule conjugate or complex that comprises a drug or pharmaceutical, or a conjugate thereof, from the blood or luminal compartment to a brain compartment.

11. The method of claim 1, wherein STX-4 expression or cellular levels of STX-4 is increased in a HBMEC compared to otherwise similar HBMCs not treated to increase STX-4 expression or STX-4 level.

12. The method of claim 1, wherein STX-4 expression or STX-4 level is increased by administering a drug, peptide or polynucleotide that increases STX-4 expression.

13. The method of claim 1, wherein STX-4 expression is increased by administering live, attenuated, or dead neonatal meningitis Escherichia coli (NMEC), Streptococcus pneumoniae, or group B Streptococcus, LPS, or components thereof which increases expression of STX-4.

14. The method of claim 1, wherein level of STX-4 in HBMECs is increased by transiently or permanently transforming the HBMECs with a nucleic acid encoding STX-4.

15. The method of claim 1, wherein STX-4 level in HBMECs is increased by loading the HBMECs with exogenous STX-4.

16. The method of claim 1, wherein STX-4 level in HBMECs is increased by loading HBMEC vesicles comprising TfR with exogenous STX-4.

17. The method of claim 1, wherein said transcytosis comprises movement of the target molecule or target molecule conjugate or complex that comprises an antibody or other protein molecule, or a conjugate or complex thereof, from the blood or luminal compartment to a brain compartment.

18. The method of claim 1, wherein said transcytosis comprises movement of the target molecule that is a drug or pharmaceutical, or a conjugate thereof, from the blood or luminal compartment to a brain compartment.

19. The method of claim 1, wherein both VAMP3 and STX-4 expression or levels are increased compared to an otherwise similar HBMCs not treated to increase VAMP3 expression or VAMP3 level and STX-4 expression or STX-4 level.

20. A composition comprising an agent that increases the expression of VAMP3 and/or STX-when administered to HBMEC cells in combination with a target molecule or target molecule conjugate or complex that binds to TfR and initiates transcytosis through a blood brain barrier comprising said HBMECs.


SEQUENCE LISTING (9 sequences)

SEQ ID NO: 1 - DNA, 2178 bp, Homo sapiens
  source 1..2178: mol_type = other DNA
gtcccacccatctccctggcctccggtcccaacttcgcttctctgctgaccctctctcgtcgccgctgccgccgccgcagctgccaaaatgtctacaggtccaactgctgccactggcagtaatcgaagacttcagcagacacaaaatcaagtagatgaggtggtggacataatgcgagttaacgtggacaaggttctggaaagagaccagaagctctctgagttagacgaccgtgcagacgcactgcaggcaggcgcttctcaatttgaaacgagcgcagccaagttgaagaggaaatattggtggaagaattgcaagatgtgggcaatcgggattactgttctggttatcttcatcatcatcatcatcgtgtgggttgtctcttcatgaagaaccagcggaactcaaaactgctgttcaagaaacctcttcaagacttttgacttagaacctgctatattatcaagcttacctactgttatctctaaaattttttttgtgttaatgtaaagttgaatttctaggaaacgtgcctttgttttttaatatgcactccaaattagaaggccggccccgtccacattttgcacagtgcctttacagatttacgtatgggctgatgaagaggccttcttaagttccagagtgctataatctagatgtaatgttgtcactaattaattgccattactcccagttagttacccttgtcatttggcattattttcagaaccacattttaaacctttgggtaatcagatttccaacttatgccttccagaaaaaaacactactgcctaacacaaatctgtgataacaacaggctgtgccttattttgataattttctgattccctagaagagaaccctctactttttgtaagcactactgactctcgctgtatttaagatgctggtgaagagcttttgctcttgcattagatttgaagatgtttacattgttgttattgttatgtatcacttgctaaaaatattgttttaatcagagataacctctttaaaaaaatttttaaagaactatggctatgaccaaagcttctattttgccaaaaagttaaataccgataaaatggccttaagtgtattcctgacagttaaattcagaaacgtgccaaatggaactcaaggtgccccttcagaattaaaatcattaccttgtgtgtgaaccttctacatcttcataggcctttcttccttttgaaaggctgtagacagtgtggctccccttctgattcagtattttgcatgggggttagagaaggtttgaggtagactctgaccgtctcataaaagagttctacccagcagttggcagattatcagctgtggactccagcatgtttctgataattatgcaagcaacaattctgtagcctcaagtaagaccacctgtgaacttgatcattatctggcccaaatatgaagataaactataactttggagtttgtttcctatttgtattcacattctgcttcctaaatcagttttctaaattatgcctgcaattaggcattggtcaggggtgaatggctcttttcacagagagtagccaaccagagacctttgctttgatatcatcaactgcagagaatgctgttgatgggaatgctggaagcagaaactttgtcatcggaaaaacttttcttgtatgcatgagactcaacatcaggatccacagcttaaagatgggaattcaggtatgaaagaaaacaggcaaggaggcactgagggagaaagacacagactttatcgctctgtggctcattgttactggaatattctaaaactcttgttcacatgctattatgacttataaagcagcaacagctgaggcgcaccaggacacagcttccatttctttaacgtctgttcccttaacatcgctgaaatgatttactgttgaagagatgccttgcggtgtggccagctgtgaggagaaagcagctggcagtgttaggacattagtccaccttcagcgcagggtctctggccgggtctgactcagaaaccttggtactcgccccttggccacagtgcccagacccatgtaacccactggctcctgcattaacccagaaatacctcgcttctatctgtgcacttagctgggaacttacccactgtaatcacctaaataaagtgtttataaacatga

SEQ ID NO: 2 - protein, 100 aa, Homo sapiens
MSTGPTAATGSNRRLQQTQNQVDEVVDIMRVNVDKVLERDQKLSELDDRADALQAGASQFETSAAKLKRKYWWKNCKMWAIGITVLVIFIIIIIVWVVSS

SEQ ID NO: 3 - DNA, 1630 bp, Homo sapiens
  source 1..1630: mol_type = other DNA
cccagcctcgggcaaggaagagacgcgaccatgtgcgcatgccccgaatttatcacggaggggcggggctgaggctgcgggagctggagcggggaagaaaagggaattccaacctgtggaaccttggggggtccccggggtcggcgccttcccattgactgtgggcggtgcaagggacggagcctctggcggctcgtgggggtgttggggtccgcagggggagggaggggagtgtcagagtgtgagcggggtacgggaattccaaatttgagggcctcccggctctggcgccggggagggagagctcaggccgccatgcgggacaggacccacgagctgagacagggggatgacagctcggacgaagaggacaaggagcgggtcgcgctggtggtgcacccgggcacggcacggctggggagcccggacgaggagttcttccacaaggtccggacaattcggcagactattgtcaaactggggaataaagtccaggagttggagaaacagcaggtcaccatcctggccacgccccttcccgaggagagtgagtgaaaccccggctgcagggcgcatgctccgccccagggattgtgggggttgtagttccacgcaggtggtggccagagtggtttgttgaggtgggggctgctgtttgggagtcttggccttctcttattcaggcatgaagcaggagctgcagaacctgcgcgatgagatcaaacagctggggagggagatccgcctgcagctgaaggccatagagccccagaaggaggaagctgatgagaactataactccgtcaacacaagaatgagaaaaacccagcatggggtcctgtcccagcaattcgtggagctcatcaacaagtgcaattcaatgcagtccgaataccgggagaagaacgtggagcggattcggaggcagctgaagatcaccaatgctgggatggtgtctgatgaggagttggagcagatgctggacagtgggcaaagcgaggtgtttgtgtccaatatcctgaaggacacgcaggtgactcgacaggccttaaatgagatctcggcccggcacagtgagatccagcagcttgaacgcagtattcgtgagctgcacgacatattcacttttctggctaccgaagtggagatgcagggggagatgatcaatcggattgagaagaacatcctgagctcagcggactacgtggaacgtgggcaggagcacgtcaagacggccctggagaaccagaagaaggcgaggaagaagaaagtcttgattgccatctgtgtgtccatcaccgtcgtcctcctagcagtcatcattggcgtcacagtggttggataatgtcgcacattgttggcactaggagcaccaggaacccagggcctggccttctctcccagcagcctggggggcagggcagagcctccagtcggaccccttcctcacactggcccctatgcagaagggcagacagttcttctggggttggcagctgctcattcatgatggcctcctccttcaggcctcaatgcctgggggaggcctgcactgtcctgattggccgggacacacggttttgtaaaaaattaaaaaacaaaaaaagagcatagaaaaaaaaaaaaaaaa

SEQ ID NO: 4 - protein, 219 aa, Homo sapiens
MKQELQNLRDEIKQLGREIRLQLKAIEPQKEEADENYNSVNTRMRKTQHGVLSQQFVELINKCNSMQSEYREKNVERIRRQLKITNAGMVSDEELEQMLDSGQSEVFVSNILKDTQVTRQALNEISARHSEIQQLERSIRELHDIFTFLATEVEMQGEMINRIEKNILSSADYVERGQEHVKTALENQKKARKKKVLIAICVSITVVLLAVIIGVTVVG

SEQ ID NO: 5 - DNA, 12 bp, synthetic construct
  source 1..12: mol_type = other DNA
aaatggacttcc

SEQ ID NO: 6 - protein, 8 aa, synthetic construct
DYKDDDDK

SEQ ID NO: 7 - DNA, 25 bp, synthetic construct
  source 1..25: mol_type = other DNA
caatgacctattagtgttttatttt

SEQ ID NO: 8 - DNA, 18 bp, synthetic construct
  source 1..18: mol_type = other DNA
ggcgcggcgcggggcaaa

SEQ ID NO: 9 - DNA, 36 bp, synthetic construct
  source 1..36: mol_type = other DNA
gacaaatacccgaaatggacttcctatttcaactac